(19) United States
(12) Patent Application Publication
(10) Pub. No.: US 2008/0063714 A1
(43) Pub. Date: Mar. 13, 2008

(54) Compositions for Encapsulation and Controlled Release

(76) Inventors: Hassan Sahouani, Hastings, MN (US); Robert A. Scherrer, White Bear Lake, MN (US); Kim M. Vogel, Lake Elmo, MN (US); Dennis E. Vogel, Lake Elmo, MN (US); Stephen W. Stein, Lino Lakes, MN (US); Brian J. Gabrio, Hugo, MN (US); Stephanie F. Bernatchez, Woodbury, MN (US); Richard H. Ferber, Fridley, MN (US); Wei Zou, Woodbury, MN (US)

Correspondence Address: 3M INNOVATIVE PROPERTIES COMPANY, PO BOX 33427, ST. PAUL, MN, 55133-3427, US

(21) Appl. No.: 10/595,051

(22) PCT Filed: Jul. 29, 2004
(86) PCT No.: PCT/US04/24429

§ 371 Date: Jan. 18, 2006

Related U.S. Application Data
(60) Provisional application No. 60/491,631, filed on Jul. 31, 2003.
(60) Provisional application No. 60/491,638, filed on Jul. 31, 2003.

(51) Int. Cl. A61K 9/16; A61K 47/12

(52) U.S. Cl. 424/484; 514/784

(57) ABSTRACT

The invention comprises compositions and methods useful for encapsulation and controlled release of guest molecules, such as drugs. Compositions of the present invention comprise a matrix comprising molecules that are non-covalently crosslinked by multi-valent cations, wherein the molecules that are non-covalently crosslinked are non-polymeric, have more than one carboxy functional group, and have at least partial aromatic or heteroaromatic character The compositions are characterized in that a guest molecule may be encapsulated within the matrix and subsequently released.

### FIELD

The present invention relates to the field of encapsulation and controlled release. In particular, the present invention relates to compositions and methods useful for encapsulation and controlled release of guest molecules, such as drugs.

### BACKGROUND OF THE INVENTION

Encapsulation and controlled release of a substance or material may be achieved by a number of methods. Typically, a polymeric coating may be used to either surround a substance or to form a mixture with a substance. Another common approach uses macroscopic structures having openings or membranes that allow for release of a substance. Encapsulation and controlled release finds broad utility, but is particularly useful in the field of controlled release drug delivery.

Many polymeric coatings operate to control release by swelling in the presence of water. This relies on the mechanism of diffusion through a swollen matrix, which can be difficult to control. Alternatively polymeric coatings or mixtures of polymers with a substance may also operate through erosion or degradation of the polymer. In either case, it can be difficult to control the release rate, since most polymers are highly polydisperse in nature. In addition, there are a limited number of polymers suitable for use in pharmaceutical applications, and a given polymer may interact with different substances in very different and unpredictable ways.

Macroscopic structures, such as osmotic pumps, control release by uptake of water from the environment into a chamber containing a substance that is forced from the chamber through a delivery orifice. This, however, requires a complex structure that needs to be prepared and filled with the substance that is to be delivered.

Protection of a drug from adverse environmental conditions may be desirable in certain drug delivery applications. The gastrointestinal tract represents one example of an environment that can interfere with the therapeutic efficacy of a drug. The ability to selectively protect a drug from certain environmental conditions, such as the low pH of the stomach, and to also be able to selectively and controllably deliver the drug under other environmental conditions, such as the neutral pH of the small intestine, is highly desirable.

Alteration of the rate at which the drug is released to a bioactive receptor (i.e., sustained or controlled drug release) may also be desirable in certain drug delivery applications. This sustained or controlled drug release may have the desirable effects of reducing dosing frequency, reducing side effects, and increasing patient compliance.

### SUMMARY OF THE INVENTION

In one aspect, the present invention provides a composition for encapsulation and controlled release comprising a water-insoluble matrix comprising a host molecule that is non-covalently crosslinked by multi-valent cations, wherein the host molecule is non-polymeric, has more than one carboxy functional group, and has at least partial aromatic or heteroaromatic character. The composition is characterized in that a guest molecule may be encapsulated within the matrix and subsequently released.

In another aspect, the present invention is a particulate composition comprising particles comprising a water-insoluble matrix comprising a host molecule that is non-covalently crosslinked by multi-valent cations, wherein the host molecule is non-polymeric, has more than one carboxy functional group, and has at least partial aromatic or heteroaromatic character. The composition is characterized in that a guest molecule may be encapsulated within the matrix and subsequently released.

The present invention can provide a matrix that will selectively protect a drug from certain environmental conditions and then controllably deliver the drug under other environmental conditions. In one aspect, the matrix will be stable in the acidic environment of the stomach and will dissolve when passed into the non-acidic environment of the intestine when administered to an animal. In another aspect, the matrix will protect a drug from enzymatic degradation.

The present invention can also provide a matrix that will effectively isolate drug molecules in a particle, such that unfavorable interactions (e.g., chemical reactions) between different drugs in a combination dosage form, unfavorable changes in a single drug component (e.g., Ostwald ripening or particle growth, changes in crystalline form), and/or unfavorable interactions between a drug and one or more excipients can be avoided. In one aspect, the matrix of the present invention would allow two drugs that are ordinarily unstable in each other's presence to be formulated into a stable dosage form. In another aspect, the matrix of the present invention would allow a drug and excipient that are ordinarily unstable in each other's presence to be formulated into a stable dosage form.

The present invention can also provide a method of preparing a matrix that will selectively protect a drug from certain environmental conditions by a process of directly mixing a host molecule, a guest molecule, and a multivalent crosslinking ion.

These and other features and advantages of the invention may be described below in connection with various illustrative embodiments of the invention.

### BRIEF DESCRIPTION OF THE DRAWINGS

FIG. 1 is a schematic drawing showing an individual host molecule and an individual multi-valent cation.

FIG. 2 is a schematic showing a water-insoluble matrix of the present invention.

FIG. 3 is a schematic showing a water-insoluble matrix of the present invention further comprising an encapsulated guest molecule.

FIG. 4 is a schematic showing dissociation of the constituents of the water-insoluble matrix and release of the guest molecule in the presence of univalent cations.

### DETAILED DESCRIPTION OF THE INVENTION

The present invention provides a composition for encapsulation and controlled release comprising a water-insoluble matrix comprising a host molecule that is non-covalently crosslinked by multi-valent cations, wherein the host molecule is non-polymeric, has more than one carboxy functional group, and has at least partial aromatic or heteroaromatic character. The composition is characterized in that a guest molecule may be encapsulated within the matrix and subsequently released.

It has now been surprisingly found that certain non-polymeric molecules containing more than one carboxy functional group can associate with multi-valent cations to form a water-insoluble matrix that is capable of encapsulating a guest molecule and that is further capable of subsequently controllably releasing the guest molecule.

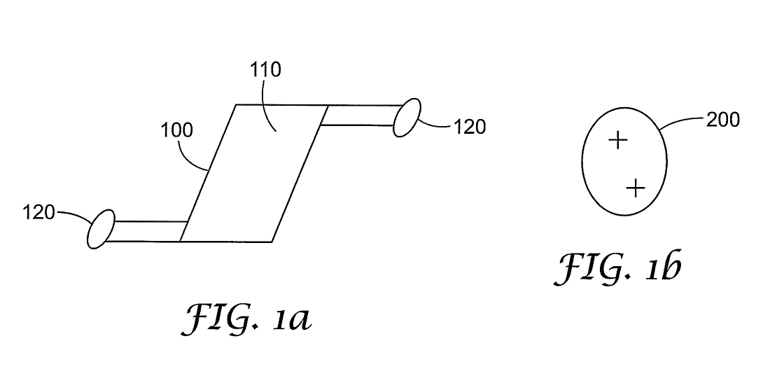

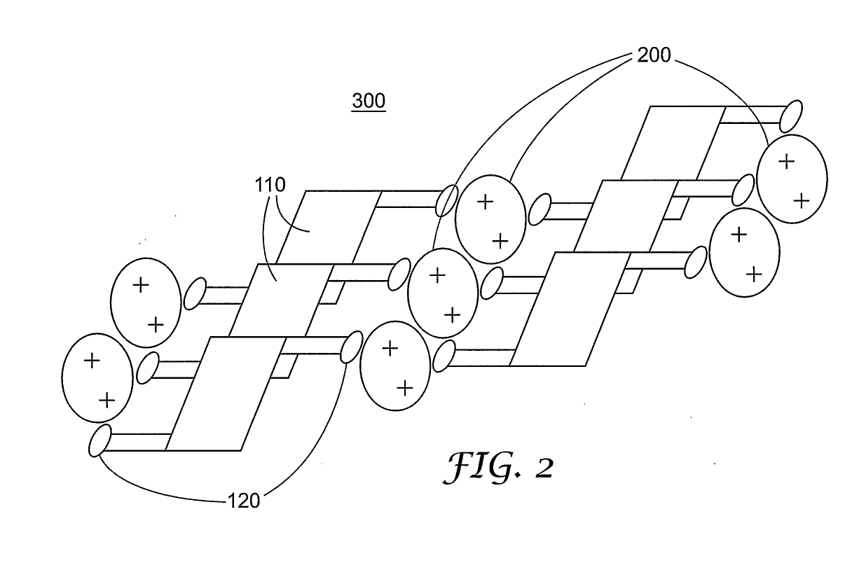

Although many morphologies may arise depending on the particular composition and amounts of the host molecules and multi-valent cations, a schematic of one embodiment is described by FIG. 1a,b and FIG. 2. FIG. 1a,b shows a schematic representation of an isolated host molecule 100 and an isolated multi-valent cation 200. The host molecule 100 has aromatic functionality 110 that is schematically represented as a planar or sheet-like area within the host molecule 100. The depicted host molecule 100 also has two carboxy functional groups 120 that are attached to the aromatic functionality 110. The multi-valent cation 200 is schematically represented by an oval. FIG. 2 shows one possible arrangement of a water-insoluble matrix 300. The aromatic functionality 110 of adjacent host molecules 100 form a layered stack of host molecules. These layered stacks have further interactions between their carboxy groups 120 and the multi-valent cations 200 which provides for linking between the layered stacks. The crosslinking of the layered stacks of host molecules is allowed because of the multiple valency of the cations. As depicted in FIG. 2, a divalent cation is able to create a non-covalent, bridging linkage between carboxy groups 120 on two different host molecules 100. Although not shown, additional valency of a cation would provide for additional non-covalent, bridging linkages between carboxy groups 120.

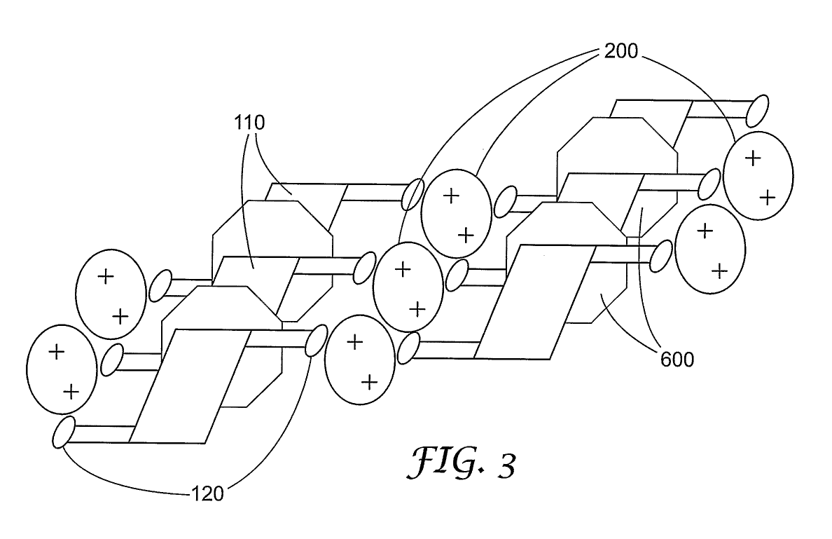

The water-insoluble matrices of the present invention are characterized in that a guest molecule may be encapsulated within the matrix and subsequently released. Encapsulation of a guest molecule 600 is shown schematically in FIG. 3, where a single guest molecule 600 is encapsulated between each pair of host molecules 100. Although the depiction in FIG. 3 shows an individual interleaving of guest and host molecules, it should be understood that the encapsulation described here may be more broadly interpreted. The guest molecule is dispersed within the matrix such that it is encapsulated. As such, the guest molecule will be effectively isolated by the matrix from an outside environment. For example, a guest molecule that is ordinarily soluble in water may be prevented from dissolving into water, since it is encapsulated within a water-insoluble matrix. Likewise, guest molecules that are unstable in the presence of an acid may be effectively isolated by the matrix. Thus, they will not degrade while encapsulated within the matrix. In one aspect, (as shown in FIG. 3) the guest molecule is intercalated in the matrix. That is, the guest molecule is present within the matrix as isolated molecules surrounded by the host molecules, rather than as aggregations of guest molecules dispersed within the matrix. Where the guest and host molecules have similar dimensions, this intercalation may take the form of an alternating structure of host and guest molecules. Where the guest molecule is substantially larger than a host molecule, several host molecules may surround a single guest molecule. Conversely, where the guest molecule is substantially smaller than a host molecule, the spacing of the matrix may be such that more than one guest molecule may be encapsulated between adjacent host molecules. More than one type of guest molecule may be encapsulated within the matrix.

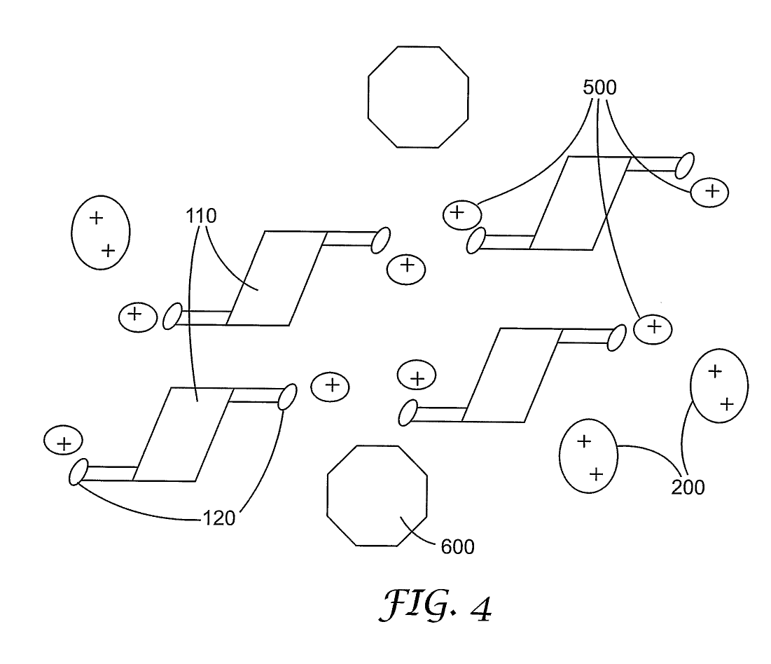

As shown in FIG. 4, if the multi-valent cations are replaced by univalent cations 500 in an aqueous solution, then the non-covalent, bridging linkages are lost, since the univalent cations will only associate with a single carboxy group 120. This allows the host molecules 100 to dissociate from each other and release the guest molecules 600. Release of a guest molecule will depend on a number of factors, including the types and amounts of guest molecules, the types and amounts of multi-valent cations present, the types and amounts of host molecules and the environment into which the matrix is placed.

The description above and in FIGS. 1-4 is intended to illustrate the general nature of the present invention, but it should be understood that the depictions are not intended to specify precise bonding interactions or detailed three-dimensional structure, and that these schematics should not be considered to be limiting to the scope of the present invention. Rather, the additional description below provides further explanation of the constituents of the present invention and their arrangement.

The water-insoluble matrix comprises a host molecule that is non-covalently crosslinked by multi-valent cations. By water-insoluble it should be understood that the matrix is essentially not soluble in substantially pure water, such as deionized or distilled water. In many instances, the matrix of the present invention will be in the form of a precipitate when present in an aqueous solution. In certain embodiments, the matrix may be in the form of a small particulate that may be suspended and/or uniformly dispersed within an aqueous solution, but this sort of dispersion is not to be equated with solubility. Furthermore, in some instances an aqueous solution may contain free host molecules and and/or free multi-valent cations that are soluble in an aqueous solution when present as isolated, or free, molecules, but these free host molecules and/or free multi-valent cations will not be in the form of the water-insoluble matrix of the invention. Under certain conditions the matrix will dissolve in cation-containing aqueous solutions, as will be evident from the description below on release of guest molecules, but this dissolution in specific cation-containing aqueous solutions is not indicative of water solubility.

The host molecule is non-polymeric, has more than one carboxy functional group, and has at least partial aromatic or heteroaromatic character. By non-polymeric, it is meant that the host molecule does not meet the standard definition of a polymer (see Handbook of Chemistry and Physics, 78^{th} ed., p. 2-51, “A substance composed of molecules of high relative molecular mass (molecular weight), the structure of which essentially comprises the multiple repetition of units derived, actually or conceptually, from molecules of low relative molecular mass.”) Although the precise definitions of high and low relative molecular mass are not specifically enumerated, for purposes of the present invention the term non-polymeric includes short chain oligomers, such as dimers, trimers, and tetramers. In one aspect, the host molecule consists of a single molecular unit, that is, it cannot be represented by repeating molecular units. Non-polymeric host molecules are typically of relatively low molecular weight when compared to typical high molecular weight polymers, and preferably have a molecular weight less than 2000 g/mol, more preferably less than 1000 g/mol, and most preferably less than 600 g/mol.

The host molecule has more than one carboxy functional group, represented in its unionized form by the chemical structure —COOH. The host molecule may have several carboxy functional groups, for example two or three carboxy functional groups, and in many cases two carboxy functional groups. The carboxy groups may be attached to adjacent carbon molecules on the host molecule (i.e., HOOC—C—C—COOH), but are usually attached to carbon molecules that are separated by one or more intervening atoms. It should be understood that the term carboxy functional group is intended to encompass free ionized forms, such as the chemical structure —COO⁻, as well as salts of carboxy functional groups (i.e., carboxylates), including, but not limited to, for example, sodium, potassium, and ammonium salts.

The host molecule is further defined in that it has at least partial aromatic or heteroaromatic character. By partial aromatic character, it is meant that at least one portion of the host molecule is characterized by a cyclic delocalized π-electron system. In general, these compounds all share the common characteristic that they have delocalized π-electrons that may be expressed by using multiple resonance structures with 4n+2 π-electrons. Aromatic as a term refers to ring structures containing only carbon, examples of which are phenyl or naphthyl groups. By partial heteroaromatic character, it is meant that at least one portion of the host molecule is characterized by a cyclic delocalized π-electron system as in the case of aromatic character, with the exception that the ring structure contains at least one atom other than carbon, for example nitrogen, sulfur, or oxygen. Examples of heteroaromatic functionalities include pyrrole, pyridine, furan, thiophene, and triazine. Host molecules preferably have more than one aromatic or heteroaromatic functional group.

In one aspect, the carboxy groups may be directly attached to an aromatic or heteroaromatic functional group (e.g., carboxyphenyl). In another aspect, when the host molecule has more than one aromatic or heteroaromatic functional group, the carboxy groups are arranged such that each aromatic or heteroaromatic group has no more than one carboxy group directly attached. Examples of such host molecules include aurintricarboxylic acid, pamoic acid, 5-{4-[[4-(3-carboxy-4-chloroanilino)phenyl](chloro)phenylmethyl]anilino}-2-chlorobenzoic acid, aluminon ammonium salt, and triazine derivatives described in U.S. Pat. No. 5,948,487 (Sahouani, et al.), the disclosure of which is incorporated by reference.

In one aspect, the host molecule contains at least one formal positive charge. In another aspect, the host molecule may be zwitterionic, that is, carrying at least one formal positive and one formal negative charge. Zwitterionic host molecules of the present invention will carry at least one negative charge. In one aspect, the negative charge will be carried through a carboxy group having a dissociated hydrogen atom, —COO⁻. The negative charge may be shared among the multiple carboxy functional groups present, such that a proper representation of the host molecule consists of two or more resonance structures. Alternatively, the negative or partial negative charges may be carried by other acid groups in the host molecule.

Triazine derivatives with the structure below are preferred host molecules.

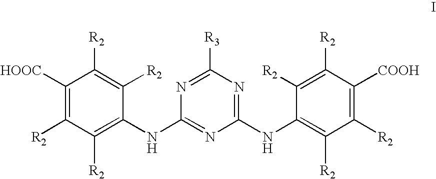

Formula I above shows an orientation of the carboxy (—COOH) group that is para with respect to the amino linkage to the triazine backbone of the compound. As depicted above the host molecule is neutral, but it may exist in alternative forms, such as a zwitterion or proton tautomer, for example where a hydrogen atom is dissociated from one of the carboxyl groups and is associated with one of the nitrogen atoms in the triazine ring. The host molecule may also be a salt. The carboxy group may also be beta with respect to the amino linkage, as shown in formula II below (or may be a combination of para and meta orientations, which is not shown).

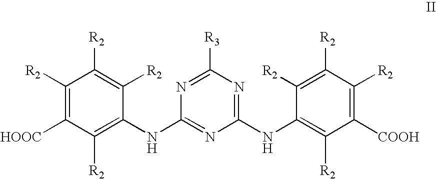

Each R₂ is independently selected from any electron donating group, electron withdrawing group and electron neutral group. Preferably, R₂ is hydrogen or a substituted or unsubstituted alkyl group. More preferably, R₂ is hydrogen, an unsubstituted alkyl group, or an alkyl group substituted with a hydroxy, ether, ester, sulfonate, or halide functional group. Most preferably R₂ is hydrogen.

R₃ may be selected from the group consisting of: substituted heteroaromatic rings, unsubstituted heteroaromatic rings, substituted heterocyclic rings, and unsubstituted heterocyclic rings, that are linked to the triazine group through a nitrogen atom within the ring of R₃. R₃ can be, but is not limited to, heteroaromatic rings derived from pyridine, pyridazine, pyrimidine, pyrazine, imidazole, oxazole, isoxazole, thiazole, oxadiazole, thiadiazole, pyrazole, triazole, triazine, quinoline, and isoquinoline. Preferably R₃ comprises a heteroaromatic ring derived from pyridine or imidazole. A substituent for the heteroaromatic ring R₃ may be selected from, but is not limited to, any of the following substituted and unsubstituted groups: alkyl, carboxy, amino, alkoxy, thio, cyano, amide, sulfonate, hydroxy, halide, perfluoroalkyl, aryl, ether, and ester. The substituent for R₃ is preferably selected from alkyl, sulfonate, carboxy, halide, perfluoroalkyl, aryl, ether, and alkyl substituted with hydroxy, sulfonate, carboxy, halide, perfluoroalkyl, aryl, and ether. When R₃ is a substituted pyridine the substituent is preferably located at the 4-position. When R₃ is a substituted imidazole the substituent is preferably located at the 3-position. Suitable examples of R₃ include, but are not limited to: 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl, shown in formulae IV to XIII below. Examples of heterocyclic rings that R₃ may be selected from include, for example, morpholine, pyrrolidine, piperidine, and piperazine.

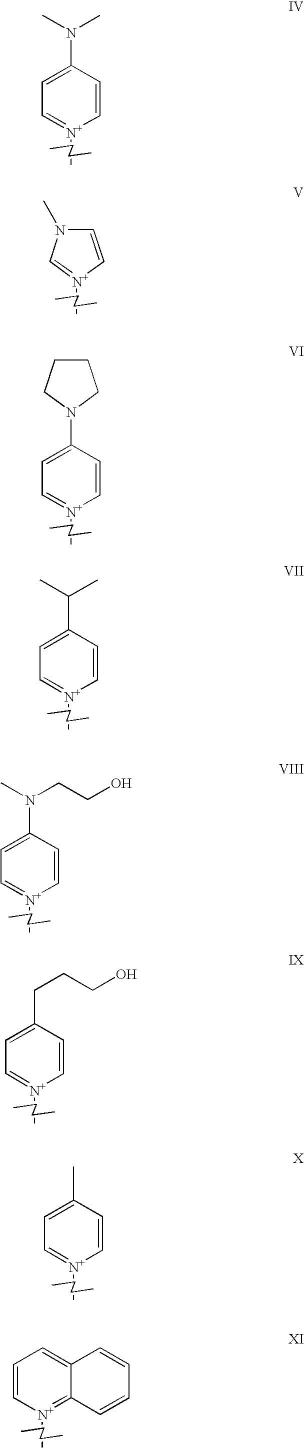

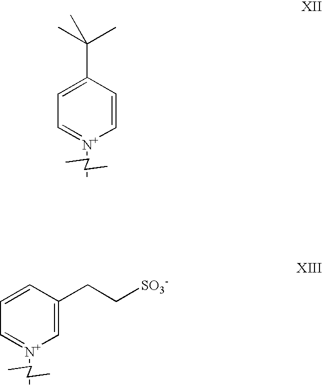

In one aspect, the R₃ group shown in formula V above may also have a substituent group other than methyl attached to the imidazole ring, as shown below,

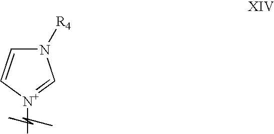

where R₄ is hydrogen or a substituted or unsubstituted alkyl group. More preferably, R₄ is hydrogen, an unsubstituted alkyl group, or an alkyl group substituted with a hydroxy, ether, ester, sulfonate, or halide functional group. Most preferably R₄ is propyl sulfonic acid, methyl, or oleyl.

As depicted above the host molecule of formula I and II is neutral, however host molecules of the present invention may exist in an ionic form wherein they contain at least one formal positive charge. In one embodiment, the host molecule may be zwitterionic. An example of such a zwitterionic host molecule, 4-{[4-(4-carboxyanilino)-6-(1-pyridiniumyl)-1,3,5-triazin-2-yl]amino}benzoate, is shown in formula III below where R₃ is a pyridine ring linked to the triazine group through the nitrogen atom of the pyridine ring. As shown, the pyridine nitrogen carries a positive charge and one of the carboxy functional groups carries a negative charge (and has a dissociated cation, such as a hydrogen atom), —COO⁻.

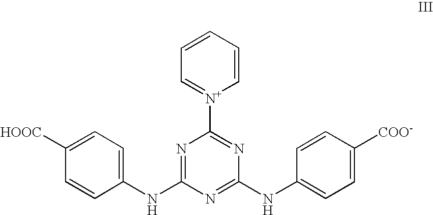

The molecule shown in formula III may also exist in other tautomeric forms, such as where both carboxy functional groups carry a negative charge and where positive charges are carried by one of the nitrogens in the triazine group and the nitrogen on the pyridine group.

As described in U.S. Pat. No. 5,948,487 (Sahouani, et al.), triazine derivatives with formula I may be prepared as aqueous solutions, or may be prepared as salts which can later be re-dissolved to form an aqueous solution. A typical synthetic route for the triazine molecules shown in I above involves a two-step process. Cyanuric chloride is treated with 4-aminobenzoic acid to give 4-{[4-(4-carboxyanilino)-6-chloro-1,3,5-triazin-2-yl]amino}benzoic acid. This intermediate is treated with a substituted or unsubstituted nitrogen-containing heterocycle. The nitrogen atom of the heterocycle displaces the chlorine atom on the triazine to form the corresponding chloride salt. The zwitterionic derivative, such as that shown in formula III above, is prepared by dissolving the chloride salt in ammonium hydroxide and passing it down an anion exchange column to replace the chloride with hydroxide, followed by solvent removal. Alternative structures, such as that shown in II above, may be obtained by using 3-aminobenzoic acid instead of 4-aminobenzoic acid.

In one embodiment, the molecules that are non-covalently crosslinked are capable of forming either a chromonic phase or assembly when dissolved in an aqueous solution before they are in the presence of multi-valent cations (i.e., before they are crosslinked). In another embodiment, the molecules that are non-covalently crosslinked are capable of forming either a chromonic phase or assembly when dissolved in an alkaline aqueous solution before they are in the presence of multi-valent cations (i.e., before they are crosslinked). Chromonic phases or assemblies are well known (see, for example, Handbook of Liquid Crystals, Volume 2B, Chapter XVIII, Chromonics, John Lydon, pp. 981-1007, 1998) and consist of stacks of flat, multi-ring aromatic molecules. The molecules consist of a hydrophobic core surrounded by hydrophilic groups. The stacking takes on a number of morphologies, but is typically characterized by a tendency to form columns created by a stack of layers. Ordered stacks of molecules are formed that grow with increasing concentration, but they are distinct from micellar phases, in that they generally do not have surfactant-like properties and do not exhibit a critical micellar concentration. Typically, the chromonic phases will exhibit isodesmic behavior, that is, addition of molecules to the ordered stack leads to a monotonic decrease in free energy. In one aspect, the molecules that are non-covalently crosslinked are host molecules that will form either a chromonic M or N phase in aqueous solution before they are in the presence of multi-valent cations (i.e., before they are crosslinked). In another aspect, the molecules that are non-covalently crosslinked are host molecules that will form either a chromonic M or N phase in an alkaline aqueous solution before they are in the presence of multi-valent cations (i.e., before they are crosslinked). The chromonic M phase typically is characterized by ordered stacks of molecules arranged in a hexagonal lattice. The chromonic N phase is characterized by a nematic array of columns, that is, there is long range ordering along the columns characteristic of a nematic phase, but there is little or no ordering amongst the columns, thus it is less ordered than the M phase. The chromonic N phase typically exhibits a schlieren texture, which is characterized by regions of varying index of refraction in a transparent medium.

The water-insoluble matrix of the present invention is comprised of host molecules that are non-covalently crosslinked by multi-valent cations. This crosslinking forms a three-dimensional matrix that is insoluble in water. By non-covalent, it is meant that the crosslinking does not involve permanently formed covalent (or chemical) bonds. That is, the crosslinking does not result from a chemical reaction that leads to a new, larger molecule, but rather results from associations of the cations with the host molecules that are strong enough to hold them together without undergoing a chemical reaction. These interactions are typically ionic in nature and can result from interaction of a formal negative charge on the host molecule with the formal positive charge of a multi-valent cation. Since the multi-valent cation has at least two positive charges, it is able to form an ionic bond with two or more host molecules, that is, a crosslink between two or more host molecules. The crosslinked, water-insoluble matrix arises from the combination of direct host molecule-host molecule interactions and host molecule-cation interactions. Divalent and/or trivalent cations are preferred. It is more preferred that a majority of the multivalent cations are divalent. Suitable cations include any divalent or trivalent cations, with calcium, magnesium, zinc, aluminum, and iron being particularly preferred.

In one aspect where the host molecules form a chromonic phase or assembly in an aqueous solution, the host molecules may form columns created from layered stacks of host molecules. The multi-valent cations provide crosslinks between these columns. Although not wishing to be bound by any particular theory, it is believed that the host molecules associate with each other through interaction of the aromatic functionality and the carboxy functionality. Alternatively, a multi-valent cation may associate with two or more host molecules, which in the case of a divalent cation forms a “dimer” that precipitates from solution and the precipitated “dimers” interact with each other through the host molecule functionality to form a water-insoluble matrix.

The composition is characterized in that a guest molecule may be encapsulated and released. Examples of useful guest molecules include dyes, cosmetic agents, fragrances, flavoring agents, and bioactive compounds, such as drugs, herbicides, pesticides, pheromones, and antifungal agents. A bioactive compound is herein defined as a compound intended for use in the diagnosis, cure, mitigation, treatment or prevention of disease, or to affect the structure or function of a living organism. Drugs (i.e., pharmaceutically active ingredients) are particularly useful guest molecules, which are intended to have a therapeutic effect on an organism. Alternatively, herbicides and pesticides are examples of bioactive compounds intended to have a negative effect on a living organism, such as a plant or pest. Although any type of drug may be employed with compositions of the present invention, particularly suitable drugs include those that are relatively unstable when formulated as solid dosage forms, those that are adversely affected by the low pH conditions of the stomach, those that are adversely affected by exposure to enzymes in the gastrointestinal tract, and those that are desirable to provide to a patient via sustained or controlled release. Examples of suitable drugs include antiinflaimmatory drugs, both steroidal (e.g., hydrocortisone, prednisolone, triamcinolone) and nonsteroidal (e.g., naproxen, piroxicam); systemic antibacterials (e.g., erythromycin, tetracycline, gentamycin, sulfathiazole, nitrofarantoin, vancomycin, penicillins such as penicillin V, cephalosporins such as cephalexin, and quinolones such as norfloxacin, flumequine, ciprofloxacin, and ibafloxacin); antiprotazoals (e.g., metronidazole); antifungals (e.g., nystatin); coronary vasodilators; calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors, and angiotensin converting enzyme inhibitors (e.g., captopril, lisinopril); other antihypertensives (e.g., propranolol); leukotriene antagonists; anti-ulceratives such as H2 antagonists; steroidal hormones (e.g., progesterone, testosterone, estradiol); local anesthetics (e.g., lidocaine, benzocaine, propofol); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, fentanyl); peptide hormones (e.g., human or animal growth hormones, LHRH); cardioactive products such as atriopeptides; proteinaceous products (e.g., insulin); enzymes (e.g., anti-plaque enzymes, lysozyme, dextranase); antinauseants; anticonvulsants (e.g., carbamazine); immunosuppressives (e.g., cyclosporine); psychotherapeutics (e.g., diazepam); sedatives (e.g., phenobarbital); anticoagulants (e.g., heparin); analgesics (e.g., acetaminophen); antimigraine agents (e.g., ergotamine, melatonin, sumatripan); antiarrhythmic agents (e.g., flecainide); antiemetics (e.g., metoclopromide, ondansetron); anticancer agents (e.g., methotrexate); neurologic agents such as anti-depressants (e.g., fluoxetine) and anti-anxiolytic drugs (e.g., paroxetine); hemostatics; and the like, as well as pharmaceutically acceptable salts and esters thereof. Proteins and peptides are particularly suitable for use with compositions of the present invention. Suitable examples include erythropoietins, interferons, insulin, monoclonal antibodies, blood factors, colony stimulating factors, growth hormones, interleukins, growth factors, therapeutic vaccines, and prophylactic vaccines. The amount of drug that constitutes a therapeutically effective amount can be readily determined by those skilled in the art with due consideration of the particular drug, the particular carrier, the particular dosing regimen, and the desired therapeutic effect. The amount of drug will typically vary from about 0.1 to about 70% by weight of the total weight of the water-insoluble matrix. In one aspect the drug is intercalated in the matrix.

In one embodiment, the guest molecule can be an antigen that may be used as a vaccine. In one embodiment, the guest molecule can be an immune response modifier compound. In a particular embodiment, both an antigen and an immune response modifier are present as guest molecules, whereby the immune response modifier compound can act as a vaccine adjuvant by activating toll-like receptors. Examples of immune response modifiers include molecules known to induce the release of cytokines, such as, e.g., Type I interferons, TNF-α, IL-1, IL-6, IL-8, IL-10, IL-12, IP-10, MIP-1, MIP-3, and/or MCP-1, and can also inhibit production and secretion of certain TH-2 cytokines, such as IL-4 and IL-5. Some IRM compounds are said to suppress IL-1 and TNF (U.S. Pat. No. 6,518,265). Examples of suitable immune response modifiers include imidazoquinolines, such as imiquimod, resiquimod, 4-amino-alpha,alpha,2-trimethyl-1H-imidazo[4,5-c]quinoline-1-ethanol hydrochloride, and compounds described in U.S. Pat. No. 4,689,338 (Gerster), U.S. Pat. No. 4,929,624 (Gerster et al.), U.S. Pat. No. 5,756,747 (Gerster), U.S. Pat. No. 5,977,366 (Gerster et al.), U.S. Pat. No. 5,268,376 (Gerster), and U.S. Pat. No. 5,266,575 (Gerster et al.) all incorporated herein by reference. Combined delivery of an immune response modifier and an antigen may elicit an enhanced cellular immune response (e.g., CTL activation) and a switch from a Th2 to Th1 immune response. In addition to treating and preventing other diseases, this type immune modulation can be used for regulating allergic responses and vaccinating against allergies.

The IRM compound(s) used as guest molecules may either be so-called small molecule IRMs, which are relatively small organic compounds (e.g., molecular weight under about 1000 daltons, preferably under about 500 daltons), or larger biologic molecules, such as oligonucleotide (e.g., CpG) type of IRMs. Combinations of such compounds may also be used. Many IRM compounds include a 2-aminopyridine fused to a five-membered nitrogen-containing heterocyclic ring. Examples of classes of small molecule IRM compounds include, but are not limited to, derivatives of imidazoquinoline amines including but not limited to amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, and thioether substituted imidazoquinoline amines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, and thioether substituted tetrahydroimidazoquinoline amines; imidazopyridine amines including but not limited to amide substituted imidazopyridines, sulfonamido substituted imidazopyridines, and urea substituted imidazopyridines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines, such as those disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,545,016; 6,545,017; 6,558,951; and 6,573,273; European Patent 0 394 026; U.S. Patent Publication No. 2002/0055517; and International Patent Publication Nos. WO 01/74343; WO 02/46188; WO 02/46189; WO 02/46190; WO 02/46191; WO 02/46192; WO 02/46193; WO 02/46749; WO 02/102377; WO 03/020889; WO 03/043572 and WO 03/045391. Additional examples of small molecule IRMs said to induce interferon (among other things), include purine derivatives (such as those described in U.S. Pat. Nos. 6,376,501, and 6,028,076), imidazoquinoline amide derivatives (such as those described in U.S. Pat. No. 6,069,149), and benzimidazole derivatives (such as those described in U.S. Pat. No. 6,387,938). 1H-imidazopyridine derivatives (such as those described in U.S. Pat. No. 6,518,265) are said to inhibit TNF and IL-1 cytokines. Other small molecule IRMs said to be TLR 7 agonists are shown in U.S. 2003/0199461 A1.

Examples of small molecule IRMs that include a 4-aminopyrimidine fused to a five-membered nitrogen-containing heterocyclic ring include adenine derivatives (such as those described in U.S. Pat. Nos. 6,376,501; 6,028,076 and 6,329,381; and in WO 02/08595).

Other IRM compounds include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. CpG7909 is a specific example. Other IRM nucleotide sequences lack CpG and are described, for example, in International Patent Publication No. WO 00/75304.

The combination of antigen and immune response modifier in compositions of the present invention, with one or the other or both present as guest molecules, may lead to improved vaccine efficacy or response. In one aspect, the combination of antigen and immune response modifier in compositions of the present invention leads to improved vaccine efficacy or response of therapeutic vaccines which require Th1 or CTL proliferation. In another aspect, improved vaccine efficacy or response may be provided by enhancing antigen presentation (e.g., via aggregated epitopes). In one aspect, improved vaccine efficacy or response may be provided by a depot effect. Particulate compositions of the present invention may be of a size comparable in dimension to pathogens that the immune system has evolved to combat and may thus be naturally targeted for uptake by antigen presenting cells. Also, compositions of the present invention may be delivered by a targetted means so as to achieve a localized delivery to a draining lymph node.

Phagocytosis of a particle containing both antigen and immune response modifier may allow for simultaneous delivery of immune response modifier and antigen to the same cell. This may enhance cross-presentation of an otherwise extracellular antigen as though it were an intracellular antigen (like a cancer or viral antigen). This may lead to improved antigen recognition, and CTL activation and proliferation, and allows for an efficient attack against infected cells.

When the guest molecule is a drug, the host molecule is generally non-therapeutic. Where the host molecule is present as a crosslinked, water-insoluble matrix it can modulate or control the release of the encapsulated drug, which will generally affect the therapeutic activity of the drug. Although this affect on therapeutic activity may be a direct result of the function of the host molecule in the present invention, the host molecule itself is usually non-therapeutic once it is released from the water-insoluble matrix. Thus, by non-therapeutic it is meant that the host-molecule has substantially no therapeutic activity when delivered to an intended organism (e.g., such as a person, mammal, fish, or plant) in the form of isolated molecules. The host molecule is preferably largely inert in relation to biological interactions with the organism and will thus serve as a carrier for the drug and as a means to control the release of the drug. The host molecule is preferably non-toxic, non-mutagenic, and non-irritating when provided in suitable amounts and dosage forms delivered to the organism.

In one aspect, the present invention can provide a particulate composition comprising particles comprising a water-insoluble matrix comprising a host molecule that is non-covalently crosslinked by multi-valent cations, wherein the host molecule is non-polymeric, has more than one carboxy functional group, and has at least partial aromatic or heteroaromatic character. The composition is characterized in that a guest molecule may be encapsulated within the matrix and subsequently released. The appropriate size and shape of the particles will vary depending on their intended use. For example, when a drug is encapsulated within the matrix, the appropriate size and shape of the particles will vary depending on the type and amount of drug dispersed within the matrix, the intended route of delivery of the particles and the desired therapeutic effect.

Although large particles (e.g., on the order of several millimeters in diameter) may be prepared, the mass median diameter of particles of the present invention is typically less than 100 μm in size, usually less than 25 μm in size, and in some cases less than 10 μm in size. In certain instances it may be desired to have particles less than 1 μm in size. In particular, these particle sizes may be desirable for oral delivery of drugs that are unstable in the intestine due to the presence of certain enzymes. Examples of such drugs include proteins, peptides, antibodies, and other biologic molecules that may be particularly sensitive to the body's enzymatic processes. In such cases, these small particles may be taken up into the intestinal wall directly, such that the particle primarily dissolves after passing the intestinal barrier, so that the amount of the sensitive drug exposed to the intestinal environment is minimized. Particles are typically spherical in their general shape, but may also take any other suitable shape, such as needles, cylinders, or plates.

The particles are dissolvable in an aqueous solution of univalent cations or other non-ionic compounds, such as surfactants. Typical univalent cations include sodium and potassium. The concentration of univalent cations needed to dissolve the particles will depend on the type and amount of the host molecules within the matrix, but for complete dissolution of the particles there should generally be at least a molar amount of univalent cations equivalent to the molar amount of carboxy groups in the matrix. In this way, there will be at least one univalent cation to associate with each carboxy group.

The rate at which a particle dissolves may also be adjusted by adjusting the type and amount of multi-valent cation used for crosslinking. Although divalent cations will be sufficient to crosslink the matrix, higher valency cations will provide additional crosslinking and lead to slower dissolution rates. In addition to valency, dissolution rate will also depend on the particular cation type. For example, a non-coordinating divalent cation, such as magnesium, will generally lead to faster dissolution than a coordinating divalent cation, such as calcium or zinc, which has an empty electron orbital capable of forming a coordination bond with a free electron pair. Different cation types may be mixed so as to give an average cation valency that is not an integer. In particular, a mixture of divalent and trivalent cations will generally cause a slower dissolution rate than a like matrix where all of the cations are divalent. In one aspect, all of the guest molecules will be released over time, but it may be desired in certain applications to have only a portion of the guest molecules be released. For instance, the type or amount of host molecule and multivalent cation may be adjusted such that the total amount of guest molecules that are released will vary depending on the environment into which they are placed. In one embodiment, the particles will not dissolve in an acidic solution, thus protecting acid sensitive guest molecules from degradation. In another, the particles will not dissolve in an acidic solution containing univalent cations, thus protecting acid sensitive guest molecules from degradation. In the particular instance where the guest molecule is a drug, two common types of general release profiles that are desired are immediate or sustained. For immediate release use it is typically desired that most of the drug will be released in a time period of less than about 4 hours, generally less than about 1 hour, often less than about 30 minutes, and in some cases less than about 10 minutes. In some instances it will desired that drug release will be nearly instantaneous, that is it will take place in a matter of seconds. For sustained (or controlled) release uses it is typically desired that most of the drug will be released over a time period greater than or equal to about 4 hours. Periods of one month or more may be desired, for example in various implantable applications. Oral sustained release dosages will generally release most of the drug over a time period of about 4 hours to about 14 days, sometimes about 12 hours to about 7 days. In one aspect it may be desired to release most of the drug over a time period of about 24 to about 48 hours. A combination of immediate and sustained release may also be desired, where for instance, a dosage provides an initial burst of release to rapidly alleviate a particular condition followed by a sustained delivery to provide extended treatment of the condition.

In some instances it may be desirable to have a pulsatile or multi-modal release of drug, such that the rate of release varies over time, for instance increasing and decreasing to match the circadian rhythm of an organism. Likewise, it may be desirable to provide a delayed release of drug, such that a dosage may be administered at a convenient time, such as just before going to sleep, but prevent release of the drug until a later time when it may be more efficacious, such as just before waking. One approach for achieving pulsatile, multi-modal, or delayed release profiles may be to mix two or more types of particles having different drug release characteristics. Alternatively, particles may be formed having two or more distinct phases, such as a core and shell, having different drug release characteristics.

Particles of the present invention that encapsulate a drug find particular use in oral dosage drug delivery. Typical oral dosage forms include solid dosages, such as tablets and capsules, but may also include other dosages administered orally, such as liquid suspensions and syrups. In one aspect, the compositions of the present invention will be particles that are stable in acidic solution and that will dissolve in an aqueous solution of univalent cations. In another aspect, the particles will be stable in the acidic environment of the stomach and will dissolve when passed into the non-acidic environment of the intestine when administered to an animal. When the particles are stable in acidic solution, the particles may generally be stable for periods of time longer than 1 hour, sometimes more than 12 hours, and may be stable for more than 24 hours when present in an acidic environment with a pH less than 7.0, for example less than about 5.0, and in some cases less than about 3.0.

For example, particles of the present invention can protect penicillin G from degradation in acidic environments. When exposed to an acidic environment, such as a solution with pH less than about 5.0, penicillin G is rapidly degraded. Penicillin G placed in a solution with a pH of about 2.0 and stored for 2 hours at 37° C. is almost completely degraded. Penicillin G may be encapsulated in particles of the present invention, such as those comprising triazine derivatives of formula I, and protected from degradation in acidic environment. For example, penicillin G encapsulated in crosslinked particles comprising 4-{[4-(4-carboxyanilino)-6-(3-methyl-1H-imidazol-3-ium-1-yl)-1,3,5-triazin-2-yl]amino}benzoate and a mixture of magnesium and aluminum cations may be exposed to an acidic solution with a pH of 2.0 for 2 hours at 37° C. Most of the penicillin remains undegraded after removal of the particles from the acidic solution and dissolution of the particles in a sodium chloride solution.

In another aspect, the mass median aerodynamic diameter of drug-containing particles is often less than 10 μm and in some cases less than 5 μm, such that the particles are respirable when delivered to the respiratory tract of an animal via the inhalation route of delivery. Delivery of particles by inhalation is well known and may be accomplished by various devices, including pressurized meter dose inhalers, for example, those described in U.S. Pat. No. 5,836,299 (Kwon, et al.), the disclosure of which is incorporated by reference; dry powder inhalers, for example, those described in U.S. Pat. No. 5,301,666 (Lerk, et al.), the disclosure of which is incorporated by reference; and nebulizers, for example, those described in U.S. Pat. No. 6,338,443 (Piper, et al.), the disclosure of which is incorporated by reference. It should be appreciated that respirable particles of the present invention may be incorporated into an inhalation dosage form using methods and processes available to one of ordinary skill in the art.

Drug-containing particles of the present invention may find further use in drug delivery dosages other than oral or inhalation, for example, by intravenous, intramuscular, or intraperitoneal injection, such as aqueous or oil solutions or suspensions; by subcutaneous injection; or by incorporation into transdermal, topical, or mucosal dosage forms, such as creams, gels, adhesive patches, suppositories, and nasal sprays. Compositions of the present invention may also be implanted or injected into various internal organs and tissues, for example, cancerous tumors, or may be directly applied to internal body cavities, such as during surgical procedures.

In one embodiment, the present invention comprises medicinal suspension formulations comprising particles of the present invention and a liquid. Particle suspensions in propellants, such as hydrofluorocarbons or other suitable propellants may find use in pressurized meter dose inhalers used for inhalation or nasal drug delivery. Particle suspensions in aqueous based media may find use in nebulizers used for inhalation or nasal drug delivery. Alternatively, particle suspensions in aqueous media may also find utility in intravenous or intramuscular delivery.

Particles may be prepared by mixing host molecules with multi-valent cations. Typically this is done by dissolving the host molecule in an aqueous solution and subsequently adding multi-valent cations to cause precipitation of the particles, or alternatively, by adding an aqueous solution of dissolved host molecules to a solution of multi-valent cations. Drugs (or other guest molecules) may be dispersed or intercalated in the matrix by adding drug to either the aqueous solution of host molecules or the multi-valent cation solution prior to precipitation. Alternatively, a drug may be dispersed or dissolved in another excipient or vehicle, such as an oil or propellant, prior to mixing with the host molecules or multi-valent cation solutions. Particles may be collected by, for example, filtration, spraying, or other means and dried to remove the aqueous carrier.

In one aspect, a guest molecule, such as a drug, may be dissolved in an aqueous surfactant-containing solution prior to introduction of the host molecule. Suitable surfactants include, for example, long chain saturated fatty acids or alcohols and mono or poly-unsaturated fatty acids or alcohols. Oleyl phosphonic acid is an example of a suitable surfactant. Although not to be bound by any particular theory, it is thought that the surfactant aids in dispersing the guest molecule so that it may be better encapsulated.

In one aspect, an alkaline compound is added to the guest molecule solution prior to introduction of the host molecule. Alternatively, an alkaline compound may be added to a host molecule solution prior to mixing the guest molecule and host molecule solutions. Examples of suitable alkaline compounds include ethanolamine, sodium or lithium hydroxide, or amines such as mono, di, triamines or polyamines. Although not to be bound by theory, it is thought that alkaline compounds aid in dissolving the host compound, particularly where the host compound is a triazine compound such as those described in formulas I and II above.

In one aspect, the present invention provides a method for preparing a composition for encapsulation and controlled release comprising combining an aqueous solution and an at least partially aromatic or heteroaromatic compound comprising more than one carboxy functional group to form a solution having a chromonic phase, and combining the solution having a chromonic phase with a solution of multi-valent ions to form a precipitated composition for drug delivery. Alternatively, compositions of the present invention may be prepared as films, coatings, or depots directly in contact with a patient. For example the multi-valent cations and the non-polymeric host molecule may be mixed together or applied consecutively to a particular site on a patient thus forming a coating or depot at the site depending on the method of application. One example of this is to form a topical coating by independently applying the multi-valent cations and the non-polymeric host molecule to the skin of a patient and allowing them to remain in contact for sufficient time to form a crosslinked matrix. Another example is to independently inject multi-valent cations and the non-polymeric host molecules into a body tissue or organ, such as a cancerous tumor, and allowing them to remain in contact for sufficient time to form a crosslinked matrix. Yet another example is to independently apply the multi-valent cations and the non-polymeric host molecules directly to an internal tissue during a surgical procedure, for example, to form a crosslinked matrix comprising an antibiotic to reduce the chance of infection after a surgical procedure.

In one aspect the invention comprises a kit for treating a patient with a composition for encapsulation and comprising a crosslinking agent comprising multi-valent cations; a host molecule agent comprising a non-polymeric host molecule having more than one carboxy functional group and at least partial aromatic or heteroaromatic character; and a drug. The kit may further comprise an applicator for applying the host molecule to the patient; an applicator for applying the crosslinking agent to the patient; and an applicator for applying the drug to the patient. The applicator for applying the host molecule, the crosslinking agent, and the drug to the patient are characterized in that the host molecule, the crosslinking agent, and the drug form a non-covalently crosslinked, water-insoluble matrix characterized in that the drug is encapsulated within the matrix and subsequently released. The crosslinking agent, host molecule agent, and drug may be present in any form suitable for being applied to a patient. Typical forms include dried or powdered, as a solution of multi-valent cations, for example as an aqueous solution, or as a cream or gel. In one aspect, the host molecule agent and the drug are present as a mixture, for instance, as a mixture in an aqueous solution.

The applicator for applying the host molecule agent to the patient, the applicator for applying the crosslinking agent to the patient, and the applicator for applying the drug to the patient may be independently selected from any method suitable for bringing each component into contact with the patient. Suitable applicators include, for example, syringes, spray pumps, brushes, roll-on applicators, and metered dose inhalers. In one embodiment, the applicator for applying the host molecule to the patient is a syringe, the applicator for applying the crosslinking agent to the patient is a syringe, and the applicator for applying the drug to the patient is a syringe. A single applicator may be used to apply one or more of the host molecule agent, the crosslinking agent, and the drug. In one embodiment, the applicator for applying both a mixture of host molecule agent and the drug, and the crosslinking agent is a dual barrel syringe. In one aspect, the dual barrel syringe is adapted to mix the mixture of host molecule agent and the drug, and the crosslinking agent as they are applied to the patient. In another aspect, the dual barrel syringe is adapted to independently apply the mixture of host molecule agent and the drug, and the crosslinking agent to the patient.

Compositions of the present invention can optionally include one or more additives such as, for example, initiators, fillers, plasticizers, cross-linkers, tackifiers, binders, antioxidants, stabilizers, surfactants, solubilizers, permeation enhancers, adhesives, viscosity enhancing agents, coloring agents, flavoring agents, and mixtures thereof.

In one aspect, the present invention comprises a method for drug delivery to an organism, such as a plant or animal. The method comprises providing a composition comprising a water-insoluble matrix comprising a host molecule that is non-covalently crosslinked by multi-valent cations and a drug encapsulated within the matrix. The host molecule is non-polymeric, has more than one carboxy functional group, and has at least partial aromatic or heteroaromatic character. The composition is delivered to an organism such that it comes into contact with univalent cations and releases the encapsulated drug and the released drug is allowed to remain in contact with a part of the organism for a period of time sufficient to achieve the desired therapeutic effect. In one embodiment, the composition is delivered to an animal orally. In another, the composition will not release the encapsulated drug until it has passed into the intestine. The encapsulated drug may be released immediately upon passing into the intestine or it may be released in a sustained fashion while residing within the intestine. In some instances, the encapsulated drug may also pass into or across the intestinal membrane and release the drug elsewhere in the animal, such as in the circulatory system. In still another embodiment, the composition is delivered via oral or nasal inhalation.

### EXAMPLES

### Preparation of Evan's Blue Color Standards

A set of 20 mL solutions to be used as color standards was prepared as follows. A solution of 0.0108 g Evan's Blue (6,6′-[Dimethyl[1,1′-biphenyl]-4,4′-diyl)bis(azo)]bis[4-amino-5-hydroxy-1,3-naphthalene disulfonic acid]tetrasodium salt), in 20 mL water was prepared. This was used as a 100% intensity color standard. Solutions of 0.0086 g, 0.0065 g, 0.0043 g, 0.0022 g, 0.0011 g Evan's Blue in 20 mL water were prepared by dilution of a 100% intensity color standard solution to prepare color standards of 80%, 60%, 40%, 20%, and 10%, respectively. A pure water sample was used as a 0% color standard. Where a solution to be compared to the color standards did not exactly match any single color standard, an estimated color was determined by interpolation.

### Example 1

A mixture was prepared by adding 6.5046 g of purified deionized water and 2.0087 g of 1-[4,6-bis(4-carboxyanilino)-1,3,5-triazin-2-yl]-3-methyl-1H-imidazol-3-ium chloride to a glass container and mixing for approximately 5 minutes. To this mixture, 0.5047 g of 1N ethanolamine was added and stirred until 1-[4,6-bis(4-carboxyanilino)-1,3,5-triazin-2-yl]-3-methyl-1H-imidazol-3-ium chloride was fully dissolved. At this step 3.0174 g of the mixture was removed and then 0.1666 g of Evan's Blue dye was added to the remaining solution and stirred until the dye fully dissolved. The concentration of Evan's Blue was 2.7% (w/w).

A 20 mL solution of 35% magnesium chloride hexahydrate in water (w/w) was prepared in a glass vial. An aliquot of 0.4 g of the Evan's Blue solution prepared above was added to the magnesium chloride solution. The resulting mixture consisted of small, precipitated beads in a clear solution. No Evan's Blue was visible in solution. The mixture was allowed to rest for 20 minutes after addition of the Evan's Blue solution, following which the solution was decanted and the beads were rinsed twice with approximately 10 ml of purified deionized water. The beads were then transferred to an empty glass vial.

### Example 2

Precipitated beads were prepared as in Example 1 with the exception that the 35% magnesium chloride hexahydrate in water solution also contained 0.1% aluminum lactate (w/w).

### Example 3

Precipitated beads were prepared as in Example 1 with the exception that the 35% magnesium chloride hexahydrate in water solution also contained 1.0% aluminum lactate (w/w).

### Example 4

Precipitated beads were prepared as in Example 1 with the exception that the 35% magnesium chloride hexahydrate in water solution was replaced by a 10% calcium chloride dihydrate solution in water (w/w).

### Example 5

Precipitated beads were prepared as in Example 4 with the exception that the 10% calcium chloride dihydrate solution in water (w/w) also contained 0.1% aluminum lactate.

### Example 6

Precipitated beads were prepared as in Example 4 with the exception that the 10% calcium chloride dihydrate solution in water (w/w) also contained 1.0% aluminum lactate.

### Example 7

Precipitated beads were prepared as in Example 4 with the exception that a 20% calcium chloride dihydrate solution in water (w/w) was used.

Release of Evan's Blue from the beads prepared in Examples 1 to 7 was measured by adding 20 mL of sodium chloride buffer solution (pH approx. 7.5) to the vial with the beads and observing the color of the resulting solution as a function of time. The % release at selected time points was estimated by comparing the solution color to the color standards prepared above and is reported in Table 1.

TABLE 1
Evan's Blue Release (% release)

| Ex. | | | | | | | | | | | | | |
|---|---|---|---|---|---|---|---|---|---|---|---|---|---|
| No. | 0 min | 1 min | 2 min | 5 min | 10 min | 25 min | 30 min | 45 min | 60 min | 90 min | 150 min | 240 min | 360 min |
| 1 | 0 | 0 | 1 | 2 | 15 | — | 40 | — | 40 | — | 40 | — | — |
| 2 | 0 | 0 | 0 | 3 | 15 | — | — | — | — | — | — | 35 | 60 |
| 3 | 0 | 0 | 0 | 2 | 5 | — | — | 15 | — | — | — | — | — |
| 4 | 0 | 0 | 9 | 10 | 25 | — | — | — | — | 38 | — | — | — |
| 5 | 0 | 0 | 0 | 7 | 20 | 90 | — | — | — | — | — | 99 | — |
| 6 | 0 | 0 | 0 | 9 | 20 | — | — | — | 90 | — | — | 99 | — |
| 7 | 0 | 0 | 8 | 10 | 30 | — | — | — | — | — | 40 | — | — |

### Example 8

A mixture was prepared by adding 5.9907 g of purified deionized water and 1.9938 g of 1-[4,6-bis(4-carboxyanilino)-1,3,5-triazin-2-yl]-3-methyl-1H-imidazol-3-ium chloride to a glass container and mixing for approximately 5 minutes. To this mixture, 0.5006 g of 1N ethanolamine was added and stirred for approximately 5 minutes. To this mixture, 0.5163 g ammonium chlorate was added and stirred until the 1-[4,6-bis(4-carboxyanilino)-1,3,5-triazin-2-yl]-3-methyl-1H-imidazol-3-ium chloride was fully dissolved. At this step 2.9820 g of the mixture was removed and then 0.1659 g of Evan's Blue dye was added to the remaining solution and stirred until the dye fully dissolved. The concentration of Evan's Blue was 2.7% (w/w).

A 20 mL solution of 35% magnesium chloride hexahydrate in water (w/w) was prepared in a glass vial. An aliquot of 0.4 g of the Evan's Blue solution prepared above was added to the magnesium chloride solution. The resulting mixture consisted of small, precipitated beads in a clear solution. No Evan's Blue was visible in solution. The mixture was allowed to rest for 20 minutes after addition of the Evan's Blue solution, following which the solution was decanted and the beads were rinsed twice with approximately 10 ml of purified deionized water. The beads were then transferred to an empty glass vial.

### Example 9

Precipitated beads were prepared as in Example 8 with the exception that the 35% magnesium chloride hexahydrate in water solution also contained 0.1% aluminum lactate (w/w).

### Example 10

Precipitated beads were prepared as in Example 8 with the exception that the 35% magnesium chloride hexahydrate in water solution also contained 1.0% aluminum lactate (w/w).

### Example 11

Precipitated beads were prepared as in Example 8 with the exception that the 35% magnesium chloride hexahydrate in water solution was replaced by a 10% calcium chloride dihydrate solution in water (w/w).

### Example 12

Precipitated beads were prepared as in Example 11 with the exception that the 10% calcium chloride dihydrate solution in water (w/w) also contained 0.1% aluminum lactate.

### Example 13

Precipitated beads were prepared as in Example 11 with the exception that the 10% calcium chloride dihydrate solution in water (w/w) also contained 1.0% aluminum lactate.

### Example 14

Precipitated beads were prepared as in Example 11 with the exception that a 20% calcium chloride dihydrate solution in water (w/w) was used.

Release of Evan's Blue from the beads prepared in Examples 8 to 14 was measured by adding 20 mL of sodium chloride buffer solution (pH approx. 7.5) to the vial with the beads and observing the color of the resulting solution as a function of time. The % release at selected time points was estimated by comparing the solution color to the color standards prepared above and is reported in Table 2.

TABLE 2
Evan's Blue Release (% release)

| Ex. | | | | | | | | | | | | | |
|---|---|---|---|---|---|---|---|---|---|---|---|---|---|
| No. | 0 min | 1 min | 2 min | 5 min | 10 min | 25 min | 30 min | 45 min | 60 min | 90 min | 150 min | 240 min | 360 min |
| 8 | 0 | 0 | 1 | 3 | 10 | — | 20 | — | 20 | — | 20 | — | — |
| 9 | 0 | 0 | 0 | 2 | 9 | — | — | — | — | — | — | 25 | 40 |
| 10 | 0 | 0 | 0 | 1 | 1 | — | — | 9 | — | — | — | — | — |
| 11 | 0 | 0 | 0 | 9 | 20 | — | — | — | — | 38 | — | — | — |
| 12 | 0 | 0 | 0 | 8 | 20 | 35 | — | — | — | — | — | 50 | — |
| 13 | 0 | 0 | 0 | 0 | 1 | — | — | — | 10 | — | — | 15 | — |
| 14 | 0 | 0 | 0 | 6 | 12 | — | — | — | — | — | 21 | — | — |

### Example 15

A mixture was prepared by adding 6.5046 g of purified deionized water and 2.0087 g of 1-[4,6-bis(4-carboxyanilino)-1,3,5-triazin-2-yl]-3-methyl-1H-imidazol-3-ium chloride to a glass container and mixing for approximately 5 minutes. To this mixture, 0.5047 g of 1N ethanolamine was added and stirred until the 1-[4,6-bis(4-carboxyanilino)-1,3,5-triazin-2-yl]-3-methyl-1H-imidazol-3-ium chloride was fully dissolved. At this step 3.0174 g of the resulting mixture and 3.6123 g of purified deionized water was added to a glass container and mixed for approximately 5 minutes. To this solution, 0.1789 g of Evan's Blue dye was added and stirred until the dye fully dissolved. The concentration of Evan's Blue was 2.6% (w/w).

A 20 mL solution of 35% magnesium chloride hexahydrate in water (w/w) was prepared in a glass vial. An aliquot of 0.4 g of the Evan's Blue solution prepared above was added to the magnesium chloride solution. The resulting mixture consisted of small, precipitated beads in a clear solution. No Evan's Blue was visible in solution. The mixture was allowed to rest for 20 minutes after addition of the Evan's Blue solution, following which the solution was decanted and the beads were rinsed twice with approximately 10 ml of purified deionized water. The beads were then transferred to an empty glass vial.

### Example 16

Precipitated beads were prepared as in Example 15 with the exception that the 35% magnesium chloride hexahydrate in water solution also contained 0.1% aluminum lactate (w/w).

### Example 17

Precipitated beads were prepared as in Example 15 with the exception that the 35% magnesium chloride hexahydrate in water solution also contained 1.0% aluminum lactate (w/w).

### Example 18

Precipitated beads were prepared as in Example 15 with the exception that the 35% magnesium chloride hexahydrate in water solution was replaced by a 10% calcium chloride dihydrate solution in water (w/w).

### Example 19

Precipitated beads were prepared as in Example 18 with the exception that the 10% calcium chloride dihydrate solution in water (w/w) also contained 0.1% aluminum lactate.

### Example 20

Precipitated beads were prepared as in Example 18 with the exception that the 10% calcium chloride dihydrate solution in water (w/w) also contained 1.0% aluminum lactate.

### Example 21

Precipitated beads were prepared as in Example 18 with the exception that a 20% calcium chloride dihydrate solution in water (w/w) was used.

Release of Evan's Blue from the beads prepared in Examples 15 to 21 was measured by adding 20 mL of sodium chloride buffer solution (pH approx. 7.5) to the vial with the beads and observing the color of the resulting solution as a function of time. The % release at selected time points was estimated by comparing the solution color to the color standards prepared above and is reported in Table 3.

TABLE 3
Evan's Blue Release (% release)

| Ex. | | | | | | | | | | | | | |
|---|---|---|---|---|---|---|---|---|---|---|---|---|---|
| No. | 0 min | 1 min | 2 min | 5 min | 10 min | 25 min | 30 min | 45 min | 60 min | 90 min | 150 min | 240 min | 360 min |
| 15 | 0 | 0 | 1 | 1 | 8 | — | 15 | — | 15 | — | 20 | — | — |
| 16 | 0 | 1 | 1 | 1 | 8 | — | — | — | — | — | — | 12 | 20 |
| 17 | 0 | 0 | 0 | 0 | 0 | — | — | 1 | — | — | — | — | — |
| 18 | 0 | 0 | 0 | 7 | 15 | — | — | — | — | 20 | — | — | — |
| 19 | 0 | 0 | 0 | 6 | 15 | 25 | — | — | — | — | — | 60 | — |
| 20 | 0 | 0 | 0 | 1 | 3 | — | — | — | 20 | — | — | 20 | — |
| 21 | 0 | 0 | 0 | 5 | 6 | — | — | — | — | — | 10 | — | — |

### Example 22

A mixture was prepared by adding 5.9907 g of purified deionized water and 1.9938 g of 1-[4,6-bis(4-carboxyanilino)-1,3,5-triazin-2-yl]-3-methyl-1H-imidazol-3-ium chloride to a glass container and mixing for approximately 5 minutes. To this mixture, 0.5006 g of 1N ethanolamine was added and stirred for approximately 5 minutes. To this mixture, 0.5163 g ammonium chlorate was added and stirred until the 1-[4,6-bis(4-carboxyanilino)-1,3,5-triazin-2-yl]-3-methyl-1H-imidazol-3-ium chloride was fully dissolved. At this step 2.9820 g of the resulting mixture and 3.6405 g of purified deionized water was added to a glass container and mixed for approximately 5 minutes. To this solution, 0.1783 g of Evan's Blue dye was added and stirred until the dye fully dissolved. The concentration of Evan's Blue was 2.6% (w/w).

A 20 mL solution of 35% magnesium chloride hexahydrate in water (w/w) was prepared in a glass vial. An aliquot of 0.4 g of the Evan's Blue solution prepared above was added to the magnesium chloride solution. The resulting mixture consisted of small, precipitated beads in a clear solution. No Evan's Blue was visible in solution. The mixture was allowed to rest for 20 minutes after addition of the Evan's Blue solution, following which the solution was decanted and the beads were rinsed twice with approximately 10 ml of purified deionized water. The beads were then transferred to an empty glass vial.

### Example 23

Precipitated beads were prepared as in Example 22 with the exception that the 35% magnesium chloride hexahydrate in water solution also contained 0.1% aluminum lactate (w/w).

### Example 24

Precipitated beads were prepared as in Example 22 with the exception that the 35% magnesium chloride hexahydrate in water solution also contained 1.0% aluminum lactate (w/w).

### Example 25

Precipitated beads were prepared as in Example 22 with the exception that the 35% magnesium chloride hexahydrate in water solution was replaced by a 10% calcium chloride dihydrate solution in water (w/w).

### Example 26

Precipitated beads were prepared as in Example 25 with the exception that the 10% calcium chloride dihydrate solution in water (w/w) also contained 0.1% aluminum lactate.

### Example 27

Precipitated beads were prepared as in Example 25 with the exception that the 10% calcium chloride dihydrate solution in water (w/w) also contained 1.0% aluminum lactate.

### Example 28

Precipitated beads were prepared as in Example 25 with the exception that a 20% calcium chloride dihydrate solution in water (w/w) was used.

Release of Evan's Blue from the beads prepared in Examples 22 to 28 was measured by adding 20 mL of sodium chloride buffer solution (pH approx. 7.5) to the vial with the beads and observing the color of the resulting solution as a function of time. The % release at selected time points was estimated by comparing the solution color to the color standards prepared above and is reported in Table 4.

TABLE 4
Evan's Blue Release (% release)

| Ex. | | | | | | | | | | | | | |
|---|---|---|---|---|---|---|---|---|---|---|---|---|---|
| No. | 0 min | 1 min | 2 min | 5 min | 10 min | 25 min | 30 min | 45 min | 60 min | 90 min | 150 min | 240 min | 360 min |
| 22 | 0 | 0 | 1 | 4 | 9 | — | 20 | — | 20 | — | 20 | — | — |
| 23 | 0 | 0 | 0 | 0 | 7 | — | — | — | — | — | — | 18 | 20 |
| 24 | 0 | 0 | 1 | 3 | 3 | — | — | 10 | — | — | — | — | — |
| 25 | 0 | 0 | 8 | 8 | 30 | — | — | — | — | 40 | — | — | — |
| 26 | 0 | 0 | 0 | 9 | 35 | 40 | — | — | — | — | — | 60 | — |
| 27 | 0 | 0 | 0 | 1 | 4 | — | — | — | 20 | — | — | 21 | — |
| 28 | 0 | 2 | 9 | 10 | 18 | — | — | — | — | — | 30 | — | — |

### Example 29

Pamoic acid, disodium salt (3.079 g) and purified deionized water (12.000 g) were added to a container and stirred for several minutes until the solid compound was fully dispersed. Ethanolamine, 1 N (5.031 g) was added until the solid compound was completely dissolved. The resulting solution was yellow. Evan's Blue Dye (0.0345 g) was added and the mixture was stirred until the dye fully dissolved. The resulting intermediate solution was purple.

Five drops of the intermediate solution were added to a 10% calcium chloride dihydrate solution forming light blue beads. After 30 minutes, the 10% calcium chloride dihydrate solution was clear. The 10% calcium chloride dihydrate solution was decanted and replaced with purified deionized water. After 30 minutes, the water was light purple. The purified deionized water was then decanted and replaced with 1% sodium chloride solution. The beads partially dissolved and the solution turned purple.

### Example 30

5-{4-[[4-(3-carboxy-4-chloroanilino)phenyl](chloro)phenylmethyl]anilino}-2-chlorobenzoic acid (3.0020 g) and purified deionized water (12.0176 g) were added to a container and stirred for several minutes until the solid compound was fully dispersed. Ethanolamine, 1 N (1.1840 g) was added until the solid compound was completely dissolved. The resulting solution was dark blue/green. Evan's Blue Dye (0.0333 g) was added and the mixture was stirred until the dye fully dissolved. The resulting intermediate solution remained dark blue/green.

Five drops of the intermediate solution were added to a 10% calcium chloride dihydrate solution forming dark blue/green beads. After 30 minutes, a small amount of blue dye was observable in the 10% calcium chloride dihydrate solution. The 10% calcium chloride dihydrate solution was decanted and replaced with purified deionized water. After 30 minutes, the water was clear. The purified deionized water was then decanted and replaced with 1% sodium chloride solution. The beads dissolved and the solution turned dark blue/green.

### Example 31

Hematoporphyrin (3.011 g) and purified deionized water (12.037 g) were added to a container and stirred for several minutes until the solid compound was fully dispersed. Ethanolamine, 1 N (0.3945 g) was added until the solid compound was completely dissolved. The resulting solution was brown/black. Evan's Blue Dye (0.033 g) was added and the mixture was stirred until the dye fully dissolved. The resulting intermediate solution was black.

Five drops of the intermediate solution were added to a 10% calcium chloride dihydrate solution forming brown beads. After 30 minutes, the 10% calcium chloride dihydrate solution was clear. The 10% calcium chloride dihydrate solution was decanted and replaced with purified deionized water. After 30 minutes, the water was clear. The purified deionized water was then decanted and replaced with 1% sodium chloride solution. The beads dissolved and the solution turned brown.

### Example 32

Aluminon ammonium salt (3.0069 g) and purified deionized water (12.0264 g) were added to a container and stirred for several minutes until the solid compound was filly dissolved. The resulting solution was red. Evan's Blue Dye (0.0337 g) was added and the mixture was stirred until the dye fully dissolved. The resulting intermediate solution was dark red.

Five drops of the intermediate solution were added to a 10% calcium chloride dihydrate solution forming red beads. After 30 minutes, the 10% calcium chloride dihydrate solution was light red. The 10% calcium chloride dihydrate solution was decanted and replaced with purified deionized water. After 30 minutes, the water was red. The purified deionized water was then decanted and replaced with 1% sodium chloride solution. The beads dissolved and the solution turned dark red/purple.

### Example 33

Aurintricarboxylic acid (3.0006 g) and purified deionized water (12.0209 g) were added to a container and stirred for several minutes until the solid compound was fully dispersed. Ethanolamine, 1 N (0.5972 g) was added until the solid compound was completely dissolved. The resulting solution was red. Evan's Blue Dye (0.0389 g) was added and the mixture was stirred until the dye fully dissolved. The resulting intermediate solution was dark red.

Five drops of the intermediate solution were added to a 10% calcium chloride dihydrate solution forming red beads. After 30 minutes, the 10% calcium chloride dihydrate solution was a transparent red in appearance. The 10% calcium chloride dihydrate solution was decanted and replaced with purified deionized water. After 30 minutes, the water remained transparent red in appearance. The purified deionized water was then decanted and replaced with 1% sodium chloride solution. The beads dissolved and the solution turned dark red/purple.

### Example 34

1H-imidazole-4,5-dicarboxylic acid (3.0161 g) and purified deionized water (12.0092 g) were added to a container and stirred for several minutes until the solid compound was fully dispersed. Ethanolamine, 1 N (3.9644 g) was added until the solid compound was completely dissolved. The resulting solution was white. Evan's Blue Dye (0.0318 g) was added and the mixture was stirred until the dye fully dissolved. The resulting intermediate solution was dark blue.

Five drops of the intermediate solution were added to a 10% calcium chloride dihydrate solution forming blue beads. After 30 minutes, the 10% calcium chloride dihydrate solution was clear. The 10% calcium chloride dihydrate solution was decanted and replaced with purified deionized water. After 30 minutes, the water was light blue. The purified deionized water was then decanted and replaced with 1% sodium chloride solution. The beads dissolved and the solution turned dark blue.

### Example 35

2,6-Naphthalenedicarboxylic acid, dipotassium salt (3.0129 g) and purified deionized water (12.0263 g) were added to a container and stirred for several minutes until the solid compound was fully dissolved. The resulting solution was white. Evan's Blue Dye (0.0339 g) was added and the mixture was stirred until the dye fully dissolved. The resulting intermediate solution was dark blue.

Five drops of the intermediate solution were added to a 10% calcium chloride dihydrate solution forming light blue/gray beads. After 30 minutes, the 10% calcium chloride dihydrate solution was clear. The 10% calcium chloride dihydrate solution was decanted and replaced with purified deionized water. After 30 minutes, the water was light blue. The purified deionized water was then decanted and replaced with 1% sodium chloride solution. The beads dissolved and the solution turned dark blue.

### Example 36

Pamoic acid (3.2300 g) and purified deionized water (12.5899 g) were added to a container and stirred for several minutes until the solid compound was fully dispersed. Ethanolamine, 1 N (0.1737 g) was added until the solid compound was completely dissolved. The resulting solution was white. Evan's Blue Dye (0.0375 g) was added and the mixture was stirred until the dye fully dissolved. The resulting intermediate solution was dark blue.

Five drops of the intermediate solution were added to a 10% calcium chloride dihydrate solution forming blue beads. After 30 minutes, the 10% calcium chloride dihydrate solution was light blue. The 10% calcium chloride dihydrate solution was decanted and replaced with purified deionized water. After 30 minutes, the water was very light blue. The purified deionized water was then decanted and replaced with 1% sodium chloride solution. The beads dissolved and the solution turned dark blue.

### Example 37

Alizarin complexone dihydrate (0.3433 g) and purified deionized water (1.7399 g) were added to a container and stirred for several minutes until the solid compound was fully dispersed. Ethanolamine, 1 N (0.2717 g) was added until the solid compound was completely dissolved. The resulting solution was orange. Evan's Blue Dye (0.0339 g) was added and the mixture was stirred until the dye fully dissolved. The resulting intermediate solution was dark purple.

Five drops of the intermediate solution were added to a 10% calcium chloride dihydrate solution forming blue beads. After 30 minutes, the 10% calcium chloride dihydrate solution was light purple. The 10% calcium chloride dihydrate solution was decanted and replaced with purified deionized water. After 30 minutes, the water remained light purple. The purified deionized water was then decanted and replaced with 1% sodium chloride solution. The beads dissolved and the solution turned dark red/purple.

### Example 38

Penicillin G, potassium salt (0.8089 g), 1-[4,6-bis(4-carboxyanilino)-1,3,5-triazin-2-yl]-3-methyl-1H-imidazol-3-ium chloride (2.0018 g), 1 N ethanolamine, (0.4705 g), and purified deionized water (6.0153 g) were mixed together to form a stock solution. Approximately 20 mL of a crosslinking solution of 35% magnesium chloride/0.5% aluminum lactate in purified deionized water was prepared in a glass vial. An aliquot of 0.3057 g of the stock solution was added dropwise to the crosslinking solution causing beads to form in the crosslinking solution. The total amount of penicillin G, potassium salt contained in the stock solution added to the crosslinking solution was 26.6 mg.

The remaining liquid in the crosslinking solution was decanted 5 minutes after addition of the stock solution to the crosslinking solution. The decanted liquid was filtered through a 0.45 μm filter and analyzed for penicillin G and benzylpenillic acid (BPA), a known degradant of penicillin-G. This is reported in Table 5 as the “Amount in Crosslinking Solution”.

Approximately 20 mL of purified deionized water was added to the beads remaining in the glass vial and gently stirred for approximately 30 seconds. The water was decanted off and filtered through a 0.45 μm filter and analyzed for penicillin G and BPA. This is reported in Table 5 as the “Amount in Water Rinse”.

Approximately 50 mL of a 2% sodium chloride solution was added to the beads remaining in the glass vial and shaken on an orbital shaker at 270 rpm. The beads were initially on the order of 2 mm in size. The dissolution of the beads was visually observed as a function of time and qualitatively reported as 3 stages of disintegration. Stage 1 was observed when the particles began to show visible signs of disintegration. Stage 2 was observed when the beads had completely broken into large particles on the order of 0.5 to 1.0 mm in size. Stage 3 was observed when no large particles remained and any remaining solid was in the form of a fine powder. Particle dissolution results are reported in Table 6 as the time (in minutes) at which each stage of disintegration was first reached.

After shaking for 60 minutes, the solution was filtered through a 0.45 μm filter and analyzed for penicillin G and BPA. This is reported in Table 5 as the “Amount in Sodium Chloride Solution”.

The total amount of penicillin G and BPA recovered and analyzed from the 3 solutions above was divided by the total amount of penicillin G contained in the stock solution added to the crosslinking solution and reported in percentage as the “Mass Balance”. The “Amount in Sodium Chloride Solution” was divided by the total amount of penicillin G and BPA recovered and analyzed from the 3 solutions above and reported in percentage as the “Encapsulation Efficiency”.

### Example 39

A stock solution and crosslinking solution were prepared as described in Example 38. An aliquot of 0.2933 g of the stock solution was added dropwise to the crosslinking solution causing beads to form in the crosslinking solution. The total amount of penicillin G, potassium salt contained in the stock solution added to the crosslinking solution was 25.5 mg.

The remaining liquid in the crosslinking solution was decanted 15 minutes after addition of the stock solution to the crosslinking solution The decanted liquid was filtered through a 0.45 μm filter and analyzed for penicillin G and benzylpenillic acid (BPA), a known degradant of penicillin-G. This is reported in Table 5 as the “Amount in Crosslinking Solution”.

Approximately 20 mL of purified deionized water was added to the beads remaining in the glass vial and gently stirred for approximately 30 seconds. The water was decanted off and filtered through a 0.45 μm filter and analyzed for penicillin G and BPA. This is reported in Table 5 as the “Amount in Water Rinse”.

Approximately 50 mL of a 2% sodium chloride solution was added to the beads remaining in the glass vial and shaken on an orbital shaker at 270 rpm. The dissolution of the beads was visually observed as a function of time. Particle dissolution results are reported in Table 6 according to the description in Example 38.

After shaking for 60 minutes, the solution was filtered through a 0.45 μm filter and analyzed for penicillin G and BPA. This is reported in Table 5 as the “Amount in Sodium Chloride Solution”.

Mass balance and encapsulation efficiency were calculated as in Example 38 and are reported in Table 5.

### Example 40

Penicillin G, potassium salt (0.8149 g), 1-[4,6-bis(4-carboxyanilino)-1,3,5-triazin-2-yl]-3-methyl-1H-imidazol-3-ium chloride (2.0055 g), ethanolamine, 1 N (0.4741 g), asparagine (0.757 g), and purified deionized water (6.0298 g) were mixed together to form a stock solution. Approximately 20 mL of a crosslinking solution of 35% magnesium chloride/0.5% aluminum lactate in purified deionized water was prepared in a glass vial. An aliquot of 0.3275 g of the stock solution was added dropwise to the crosslinking solution causing beads to form in the crosslinking solution. The total amount of penicillin G, potassium salt contained in the stock solution added to the crosslinking solution was 26.5 mg.

The remaining liquid in the crosslinking solution was decanted 5 minutes after addition of the stock solution to the crosslinking solution. The decanted liquid was filtered through a 0.45 μm filter and analyzed for penicillin G and benzylpenillic acid (BPA), a known degradant of penicillin-G. This is reported in Table 5 as the “Amount in Crosslinking Solution”.

Approximately 20 mL of purified deionized water was added to the beads remaining in the glass vial and gently stirred for approximately 30 seconds. The water was decanted off and filtered through a 0.45 μm filter and analyzed for penicillin G and BPA. This is reported in Table 5 as the “Amount in Water Rinse”.

Approximately 50 mL of a 2% sodium chloride solution was added to the beads remaining in the glass vial and shaken on an orbital shaker at 270 rpm. The dissolution of the beads was visually observed as a function of time. Particle dissolution results are reported in Table 6 according to the description in Example 38.

After shaking for 60 minutes, the solution was filtered through a 0.45 μm filter and analyzed for penicillin G and BPA. This is reported in Table 5 as the “Amount in Sodium Chloride Solution”.

Mass balance and encapsulation efficiency were calculated as in Example 38 and are reported in Table 5.

### Example 41

A stock solution and crosslinking solution were prepared as described in Example 40. An aliquot of 0.3036 g of the stock solution was added dropwise to the crosslinking solution causing beads to form in the crosslinking solution. The total amount of penicillin G, potassium salt contained in the stock solution added to the crosslinking solution was 24.5 mg.

The remaining liquid in the crosslinking solution was decanted 15 minutes after addition of the stock solution to the crosslinking solution The decanted liquid was filtered through a 0.45 μm filter and analyzed for penicillin G and benzylpenillic acid (BPA), a known degradant of penicillin-G. This is reported in Table 5 as the “Amount in Crosslinking Solution”.

Approximately 20 mL of purified deionized water was added to the beads remaining in the glass vial and gently stirred for approximately 30 seconds. The water was decanted off and filtered through a 0.45 μm filter and analyzed for penicillin G and BPA. This is reported in Table 5 as the “Amount in Water Rinse”.

Approximately 50 mL of a 2% sodium chloride solution was added to the beads remaining in the glass vial and shaken on an orbital shaker at 270 rpm. The dissolution of the beads was visually observed as a function of time. Particle dissolution results are reported in Table 6 according to the description in Example 38.

After shaking for 60 minutes, the solution was filtered through a 0.45 μm filter and analyzed for penicillin G and BPA. This is reported in Table 5 as the “Amount in Sodium Chloride Solution”.

Mass balance and encapsulation efficiency were calculated as in Example 38 and are reported in Table 5.

TABLE 5
Encapsulation and Release of Penicillin G

| | | | | | Amount in | | | |
|---|---|---|---|---|---|---|---|---|
| | Amount in | | Amount in | | Sodium | | | |
| | Crosslinking | | Water Rinse | | Chloride | | Encapsulation | Mass |
| Ex. | Solution [mg] | | [mg] | | Solution [mg] | | Efficiency | Balance |
| No. | Pen G | BPA | Pen G | BPA | Pen G | BPA | [%] | [%] |
| 38 | 0.0 | 1.4 | 4.7 | 0.1 | 17.4 | 0.0 | 73.7 | 88.7 |
| 39 | 0.0 | 0.6 | 2.4 | 0.1 | 21.5 | 0.0 | 87.4 | 92.9 |
| 40 | 0.0 | 1.5 | 2.3 | 0.1 | 18.2 | 0.0 | 82.4 | 90.1 |
| 41 | 0.0 | 2.0 | 2.8 | 0.1 | 20.5 | 0.0 | 80.7 | 99.5 |

TABLE 6
Penicillin G bead dissolution [minutes]

| | Ex. 38 | Ex. 39 | Ex. 40 | Ex. 41 |
|---|---|---|---|---|
| Stage 1 | 5 | 5 | 7 | 7 |
| Stage 2 | 8 | 15 | 30 | 30 |
| Stage 3 | 20 | 15 | 35 | 54 |

### Example 42

A stock solution was prepared by adding deionized water (18 g), 1-[4,6-bis(4-carboxyanilino)-1,3,5-triazin-2-yl]-4-(dimethylamino)pyridinium chloride (2 g), and N-ethyl diisopropylamine (0.05 g) to a glass vial and mixing. An additional drop of N-ethyl diisopropylamine was added to the vial and the mixture was stirred until all of the solids dissolved. The pH of the stock solution was adjusted to 7.4 by addition of hydrochloric acid.

An aliquot (5 g) of the stock solution and adenosine deaminase (0.020 g, Sigma, lot no. 70H8145) were mixed in a glass vial until the adenosine deaminase was fully dissolved to prepare an intermediate solution.

A 10% calcium chloride solution in water was adjusted to a pH of 5.24 with hydrochloric acid for use as a crosslinking solution.

A portion of the crosslinking solution was placed in a glass vial and an aliquot of the intermediate solution was added dropwise to form crosslinked beads. The crosslinking solution was decanted and discarded. The remaining crosslinked beads were washed with 10 mL deionized water for approximately 10 seconds. The water was then decanted and discarded. The washed, crosslinked beads were divided into two approximately equal portions for further testing.

One portion of the beads was added to a vial containing 20 ml of a 0.1% trifluoroacetic acid in water (pH of 2.0) test solution. The beads were exposed to the acidic test solution at room temperature for two hours. The acidic test solution was then decanted and discarded. The beads were rinsed with 10 mL deionized water. The water was then decanted and discarded. Phosphate buffer (20 mL, pH of 7.0 with 0.15 M NaCl) was added to the vial with the remaining beads and the vial was agitated on a wrist action shaker for one hour to dissolve the beads. The resulting solution was filtered through a 0.22 μm poly(vinylidene fluoride) filter.

Adenosine deaminase activity was determined by mixing the filtered solution with 1.35 mM adenosine solution (pH of 7.0) in a 1:1 ratio and then incubating in a 30° C. water bath for 2 minutes. The resulting solution was then analyzed for inosine concentration by high performance liquid chromatography (Column: Hypercarb, 100×4.6 mm; Mobile phase, A=Water, B=Acetonitrile, gradient, 0 min=25% B, 5 min=25% B, 10 min=95% B; Flow Rate: 1 mL/min; Detector: UV at 215 and 260 nm; Injection Volume: 10 μL; Run time: 15 minutes). The inosine peak area was 733 units.

The other portion of the beads was added to a vial containing 20 mL of deionized water (pH approx. 7.5). The beads were exposed to the water solution for two hours. The water was then decanted and discarded. Phosphate buffer (20 mL, pH of 7.0 with 0.15 M NaCl) was added to the vial with the remaining beads and the vial was agitated on a wrist action shaker for one hour to dissolve the beads. The resulting solution was filtered through a 0.22 μm poly(vinylidene fluoride) filter. Adenosine deaminase activity was determined as described above. The inosine peak area was 812 units.

### Comparative Example

Adenosine deaminase was added to a 20 mL of 0.1% trifluoroacetic acid in water (pH of 2.0) solution to prepare an acidic test solution with a concentration of approximately 110 μg/mL adenosine deaminase. The solution was stored at room temperature for 2 hours and subsequently adjusted to a pH of 7.0 by addition of 1 N sodium hydroxide. Adenosine deaminase activity was determined as described above. The inosine peak area was 5 units.

### Example 43

All glassware and stir bars used were passivated by treating for ten minutes with an insulin solution (0.001 g insulin per 100 g purified deionized water). Bovine insulin (0.143 g, Sigma Aldrich Company) was added to purified deionized water (8.0113 g) containing oleyl phosphonic acid sodium salt (0.005 g) and ethanolamine (0.023 g) and mixed for 10 minutes. To this mixture, 1.0051 g of 1-[4,6-bis(4-carboxyanilino)-1,3,5-triazin-2-yl]-3-methyl-1H-imidazol-3-ium was added, followed by 0.1012 g ethanolamine to prepare a chromonic solution. The above mixture was stirred until the 1-[4,6-bis(4-carboxyanilino)-1,3,5-triazin-2-yl]-3-methyl-1H-imidazol-3-ium dissolved. The resulting insulin solution had a chromonic phase.

A crosslinking solution was prepared by adding calcium chloride (0.9973 g) and zinc chloride (0.0049 g) to purified deionized water (9.0018 g).

Drops of the insulin solution were released into the crosslinking solution forming beads. The formed beads were left to further crosslink for 30 minutes.

The solution was decanted from the beads and analyzed to determine the concentration of insulin that was not contained within the beads. The remaining amount of insulin is reported as the amount encapsulated within the beads. The amount encapsulated divided by the total amount added is reported as the encapsulation efficiency. The encapsulation efficiency was 93%. The beads were resuspended in Tris buffer, micronized with a tissue tearer for 30 seconds at high speed, and then allowed to sit for 1 hour at which time the solution was centrifuged and the supernatant analyzed for insulin concentration. The micronized beads were again resuspended in Tris buffer and this process was repeated at time points of 2, 3, and 4 hours to measure insulin release. At each time point, the sample was centrifuged before decanting the solution for analysis. Insulin concentration was analyzed by high performance liquid chromatography (Column: ProntoSIL C-18 300A, 150×2.0 mm; Mobile phase, A=Water with 0.1% trifluoroacetic acid, B=Acetonitrile with 0.1% trifluoroacetic acid, gradient, 0 min=20% B, 10 min=50% B, 10.01 min=95% B; Flow Rate: 1 mL/min; Detector: UV at 210 and 280 nm; Injection Volume: 5 μL; Run time: 15 minutes). Results are shown in Table 7.

TABLE 7
Insulin release [hours]

| | 1 | 2 | 3 | 4 |
|---|---|---|---|---|
| % released | 3.9 | 24.9 | 31.9 | 39.6 |

### Example 44

A solution was prepared by mixing 1-[4,6-bis(4-carboxyanilino)-1,3,5-triazin-2-yl]-3-methyl-1H-imidazol-3-ium chloride (1.0 g) with ethanolamine (0.12 g) and purified deionized water (9.0 g). To this solution, an IRM compound 4-amino-alpha,alpha,2-trimethyl-1H-imidazo[4,5-c]quinoline-1-ethanol hydrochloride (0.05 g) and ovalbumin (10 mL of 50 mg/mL solution, 0.5 g solids) were added and stirred until the IRM and ovalbumin dissolved. The resulting IRM-ovalbumin solution had a chromonic phase.

A crosslinking solution was prepared by adding magnesium chloride hexahydrate (7.0 g) to purified deionized water (13.0 g).

Drops of the IRM-ovalbumin solution (0.537 g total) were released into 15 mL of crosslinking solution thereby forming beads. The formed beads were left to further crosslink for 30 minutes.

The liquid from the solution with beads was decanted and analyzed for IRM and ovalbumin content. The results are reported in Table 8 below as “step 1” content. The beads were subsequently washed with 10 mL purified deionized water. The wash fluid was decanted from the beads and analyzed for IRM and ovalbumin content. The results are reported in Table 8 below as “wash” content. 20 mL of a 0.9% NaCl buffer solution (pH=7.0, 50 mM phosphate buffer) was then added to the beads and the resulting suspension was stored at 4° C. for approximately 3 days. The solution was then filtered through a 0.22 μm PVDF syringe filter before injection into an HPLC. The concentration of the filtered solution was analyzed for IRM and ovalbumin content. The results are reported in Table 8 below as “encapsulated” content. The percent encapsulation of the IRM and ovalbumin is reported as the percentage of each in the “bead” content divided by the total amount measured in the “step 1”, “wash”, and “bead” measurements.

IRM concentration was analyzed by high performance liquid chromatography (Column: ProntoSIL C-18, 150×3.0 mm; Mobile phase, A=Water with 0.1% formic acid, B=Acetonitrile, gradient, 0 min=10% B, 10 min=40% B, 15 min=95% B; Flow Rate: 0.5 mL/min; Detector: UV at 254 nm; Injection Volume: 2 μL; Run time: 18 minutes). Ovalbumin concentration was analyzed by high performance liquid chromatography (Column: Tosoh SW2000 aqueous GPC, 300×4.6 mm; Mobile phase, isocratic 50 mM phosphate buffer pH 7.0 0.15 M NaCl; Flow Rate: 0.35 mL/min; Detector: UV at 215 nm; Injection Volume: 10 μL; Run time: 30 minutes).

TABLE 8
IRM-ovalbumin encapsulation

| | IRM [μg] | Ovalbumin [μg] |
|---|---|---|
| Step 1 | 71 | * |
| Wash | 16 | 73 |
| Encapsulated | 2530 | 1456 |
| % Encapsulated | 96.6% | 95.2% |

*below limit of quantitation of 30 μg

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow. The complete disclosures of the patents, patent documents and publications cited herein are incorporated by reference in their entirety as if each were individually incorporated. In case of any conflict, the present specification, including definitions, shall control.

1. A composition comprising: a matrix comprising molecules that are non-covalently crosslinked by multi-valent cations, wherein the molecules that are non-covalently crosslinked are non-polymeric, have more than one carboxy functional group, and have at least partial aromatic or heteroaromatic character.

2. A composition for encapsulation and controlled release comprising a composition according to claim 1 wherein the molecules that are non-covalently crosslinked are host molecules and the composition is characterized in that a guest molecule may be encapsulated within the matrix and subsequently released.

3. A composition for encapsulation and controlled release according to claim 2, wherein the host molecule is zwitterionic.

4. A composition for encapsulation and controlled release according to claim 2, further comprising a guest molecule.

5. A composition for encapsulation and controlled release according to claim 4, wherein the guest molecule is a drug.

6. A composition according to claim 1, wherein the molecules that are non-covalently crosslinked are capable of forming either a chromonic M or N phase in aqueous solution before they are in the presence of multi-valent cations.

7. A composition according to claim 1, wherein the molecules that are non-covalently crosslinked have at least partial aromatic character.

8. A composition according to claim 1, wherein at least one of the carboxy groups of the molecules that are non-covalently crosslinked are directly attached to an aromatic or heteroaromatic functional group.

9. A composition according to claim 1, wherein a majority of the multi-valent cations are divalent.

10. A composition according to claim 1, wherein the multi-valent cations are selected from the group consisting of calcium, magnesium, zinc, aluminum, and iron.

11. A composition according to claim 1, wherein the molecules that are non-covalently crosslinked comprise: 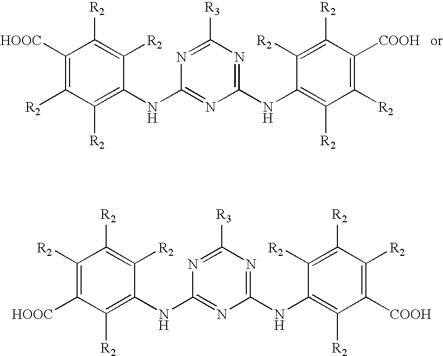
wherein
each R₂ is independently selected from any electron donating group, electron withdrawing group and electron neutral group; and
R₃ is selected from the group consisting of substituted and unsubstituted heteroaromatic and heterocyclic rings linked to the triazine group through a nitrogen atom within the ring of R₃ and proton tautomers and salts thereof.

12. A composition according to claim 11, wherein each R₂ is independently selected from the group consisting of hydrogen, an unsubstituted alkyl group, or an alkyl group substituted with a hydroxy, ether, ester, sulfonate, or halide functional group.

13. A composition according to claim 12, wherein R₃ comprises a heteroaromatic ring derived from the group consisting of pyridine, pyridazine, pyrimidine, pyrazine, imidazole, oxazole, isoxazole, thiazole, oxadiazole, thiadiazole, pyrazole, triazole, triazine, quinoline, and isoquinoline.

14. A composition according to claim 12, wherein R₃ comprises a heteroaromatic ring derived from pyridine or imidazole.

15. A composition according to claim 12, wherein R₃ is selected from the group consisting of pyridinium-1-yl, 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl.

16. A composition according to claim 11 wherein the host molecule comprises: 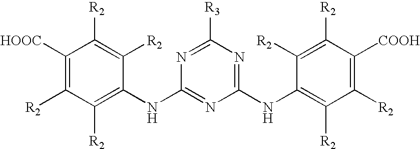
and proton tautomers and salts thereof.

17. A composition according to claim 16, wherein each R₂ is independently selected from the group consisting of hydrogen, an unsubstituted alkyl group, or an alkyl group substituted with a hydroxy, ether, ester, sulfonate, or halide functional group.

18. A composition according to claim 17, wherein R₃ comprises a heteroaromatic ring derived from the group consisting of pyridine, pyridazine, pyrimidine, pyrazine, imidazole, oxazole, isoxazole, thiazole, oxadiazole, thiadiazole, pyrazole, triazole, triazine, quinoline, and isoquinoline.

19. A composition according to claim 17, wherein R₃ comprises a heteroaromatic ring derived from pyridine or imidazole.

20. A composition according to claim 17, wherein R₃ is selected from the group consisting of pyridinium-1-yl, 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl.

21. A particulate composition comprising particles comprising a water-insoluble matrix comprising a host molecule that is non-covalently crosslinked by multi-valent cations, wherein the host molecule is non-polymeric, has more than one carboxy functional group, and has at least partial aromatic or heteroaromatic character, and the particles are characterized in that a guest molecule may be encapsulated within the matrix and subsequently released.

22. A particulate composition according to claim 21, wherein the particles are dissolvable in an aqueous solution of univalent cations.

23. A particulate composition according to claim 21, wherein the particles do not substantially dissolve in a solution with a pH less than about 5.0.

24. A particulate composition according to claim 21, wherein the mass median diameter of the particles is less than 100 μm.

25. A particulate composition according to claim 21, wherein the host molecule is zwitterionic.

26. A particulate composition according to claim 21, wherein the host molecule has two carboxy functional groups.

27. A particulate composition according to claim 21, further comprising a guest molecule.

28. A particulate composition according to claim 27, wherein the guest molecule is a drug.

29. A particulate composition according to claim 21, wherein the host molecule is capable of forming either a chromonic M or N phase in aqueous solution before it is in the presence of multi-valent cations.

30. A particulate composition according to claim 21, wherein the host molecule has at least partial aromatic character.

31. A particulate composition according to claim 21, wherein at least one of carboxy groups of the host molecule is directly attached to an aromatic or heteroaromatic functional group.

32. A particulate composition according to claim 21, wherein a majority of the multi-valent cations are divalent.

33. A particulate composition according to claim 21, wherein the multi-valent cations are selected from the group consisting of calcium, magnesium, zinc, aluminum, and iron.

34. A particulate composition according to claim 21, wherein the host molecule comprises: 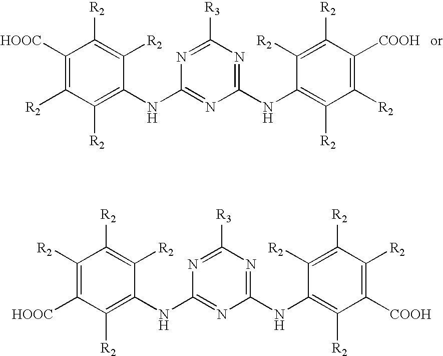
wherein
each R₂ is independently selected from any electron donating group, electron withdrawing group and electron neutral group; and
R₃ is selected from the group consisting of substituted and unsubstituted heteroaromatic and heterocyclic rings linked to the triazine group through a nitrogen atom within the ring of R₃, and proton tautomers and salts thereof.

35. A particulate composition according to claim 34, wherein each R₂ is independently selected from the group consisting of hydrogen, an unsubstituted alkyl group, or an alkyl group substituted with a hydroxy, ether, ester, sulfonate, or halide functional group.

36. A particulate composition according to claim 35, wherein R₃ comprises a heteroaromatic ring derived from the group consisting of pyridine, pyridazine, pyrimidine, pyrazine, imidazole, oxazole, isoxazole, thiazole, oxadiazole, thiadiazole, pyrazole, triazole, triazine, quinoline, and isoquinoline.

37. A particulate composition according to claim 35, wherein R₃ comprises a heteroaromatic ring derived from pyridine or imidazole.

38. A particulate composition according to claim 35, wherein R₃ is selected from the group consisting of pyridinium-1-yl, 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl.

39. A particulate composition according to claim 34 wherein the host molecule comprises: 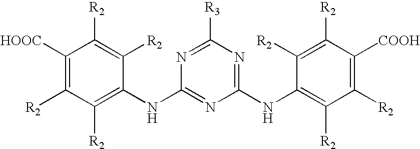
and proton tautomers and salts thereof.

40. A particulate composition according to claim 39, wherein each R₂ is independently selected from the group consisting of hydrogen, an unsubstituted alkyl group, or an alkyl group substituted with a hydroxy, ether, ester, sulfonate, or halide functional group.

41. A particulate composition according to claim 40, wherein R₃ comprises a heteroaromatic ring derived from the group consisting of pyridine, pyridazine, pyrimidine, pyrazine, imidazole, oxazole, isoxazole, thiazole, oxadiazole, thiadiazole, pyrazole, triazole, triazine, quinoline, and isoquinoline.

42. A particulate composition according to claim 40, wherein R₃ comprises a heteroaromatic ring derived from pyridine or imidazole.

43. A particulate composition according to claim 30, wherein R₃ is selected from the group consisting of pyridinium-1-yl, 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl.

44. A medicinal suspension formulation comprising a particulate composition according to claim 21 and a liquid.

45. A method for preparing a composition for encapsulation and controlled release comprising:
(a) combining an aqueous solution and an at least aromatic or heteroaromatic compound comprising more than one carboxy functional group to form a solution having a chromonic phase; and
(b) combining the solution having a chromonic phase with a solution of multi-valent ions to form a precipitated composition.

46. A method for preparing a composition for encapsulation and controlled release according to claim 45, wherein the precipitated composition further comprises a bioactive compound.

47. A method for drug delivery comprising:
(a) providing a composition comprising a water-insoluble matrix comprising:
(i) a host molecule that is non-covalently crosslinked by multi-valent cations, wherein the host molecule is non-polymeric, has more than one carboxy functional group, and has at least partial aromatic or heteroaromatic character, and
(ii) a drug encapsulated within the matrix;
(b) delivering the composition to an organism such that it comes into contact with univalent cations and releases the encapsulated drug; and
(c) allowing the released drug to remain in contact with a part of the organism for a period of time sufficient to achieve the desired therapeutic effect.

48. A method for drug delivery according to claim 47, wherein the composition is delivered to an animal orally.

49. A method for drug delivery according to claim 48, wherein encapsulated drug is delivered to the intestine.

50. A method for drug delivery according to claim 47, wherein encapsulated drug is delivered to systemic circulation prior to release.

51. A method for drug delivery according to claim 47, wherein the composition is delivered to an animal via inhalation.

52. A method for drug delivery according to claim 47, wherein the composition is delivered to an animal intravenously or intramuscularly.

53. A method of providing a drug delivery composition for encapsulation and controlled release comprising:
(i) administering a crosslinking agent comprising multi-valent cations;
(ii) administering a host molecule agent comprising a non-polymeric host molecule having more than one carboxy functional group and at least partial aromatic or heteroaromatic character; and
(iii) administering a drug;
wherein the crosslinking agent, and the drug form a non-covalently crosslinked, water-insoluble matrix and the drug is encapsulated within the matrix and subsequently released.

54. The method of claim 53, wherein at least one of the ingredients is administered independently of the others and the composition subsequently forms at a desired site for delivery.
